SEQUENCE LISTING (28 sequences)

SEQ ID NO: 1 - DNA, 3183 bp, Artificial Sequence
  Whole nucleotide sequence of the chimeric antigen receptor (CAR) capable of binding to BCMA used in the Examples
atgctgctgctcgtgacatctctgctgctgtgcgagctgccccaccccgcctttctgctgattcctcaggtgcagctggtgcagtctggcgccgaagtgaagaaaccaggcgccagcgtgaaggtgtcctgcaaggccagcggctacagcttccccgactactacatcaactgggtgcgccaggcccctggacagggcctggaatggatgggctggatctacttcgccagcggcaactccgagtacaaccagaaattcaccggcagagtgaccatgacccgggacaccagcatcaacaccgcctacatggaactgagcagcctgaccagcgaggataccgccgtgtacttctgcgccagcctgtacgactacgactggtacttcgacgtgtggggccagggcacaatggtcaccgtgtctagcggaggcggaggctccggagggggaggatctgggggaggcggaagcgatatcgtgatgacccagacccccctgagcctgagcgtgacacctggacagcctgccagcatcagctgcaagagcagccagagcctggtgcacagcaacggcaacacctacctgcactggtatctgcagaagcccggccagagcccccagctgctgatctacaaggtgtccaaccggttcagcggcgtgcccgacagattttctggcagcggctccggcaccgacttcaccctgaagatctcccgggtggaagccgaggacgtgggcatctactactgcagccagtccagcatctacccctggaccttcggccaggggaccaagctggaaatcaaaaaagagtctaagtacggaccgccttgtcctccttgtccagctcctcctgtggccggacctagcgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttccagagcacctaccgggtggtgtccgtgctgacagtgctgcaccaggactggctgaacggcaaagagtacaagtgcaaggtgtccaacaagggcctgcccagcagcatcgagaaaaccatcagcaaggccaagggccagcctcgcgagccccaggtgtacacactgcctccaagccaggaagagatgaccaagaaccaggtgtccctgacctgtctcgtgaagggcttctaccccagcgacattgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaagatgttctgggtgctggtggtcgtgggcggagtgctggcctgttacagcctgctcgtgaccgtggccttcatcatcttttgggtcaagcggggcagaaagaagctgctgtatatcttcaagcagcccttcatgcggcccgtgcagaccacacaggaagaggacggctgctcctgccggttccccgaggaagaagaaggcggctgcgagctgagagtgaagttcagcagaagcgccgacgcccctgcctatcagcagggccagaaccagctgtacaacgagctgaacctgggcagacgggaagagtacgacgtgctggataagcggagaggccgggaccctgagatgggcggcaagcctagaagaaagaacccccaggaaggcctgtataacgaactgcagaaagacaagatggccgaggcctacagcgagatcggaatgaagggcgagcggagaagaggcaagggccacgatggcctgtaccagggactgagcaccgccaccaaggatacctatgacgcactgcacatgcaggccctgccccccagactcgagggcggaggcgaaggcagaggatctctgctgacatgcggcgacgtggaagagaaccctggccccagaatgctgctgctcgtgacaagcctgctgctgtgcgagctgccccaccctgcctttctgctgatcccccggaaagtgtgcaacggcatcggcatcggagagttcaaggacagcctgtccatcaacgccaccaacatcaagcacttcaagaattgcaccagcatcagcggcgacctgcacatcctgccagtggcctttagaggcgacagcttcacccacacccccccactggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 2 - DNA, 66 bp, Artificial Sequence
  Nucleotide sequence of the GMCSF signal peptide
atgctgctgctcgtgacatctctgctgctgtgcgagctgccccaccccgcctttctgctgattcct

SEQ ID NO: 3 - DNA, 357 bp, Artificial Sequence
  Nucleotide sequence of the BCMA single chain variable fragment VH
caggtgcagctggtgcagtctggcgccgaagtgaagaaaccaggcgccagcgtgaaggtgtcctgcaaggccagcggctacagcttccccgactactacatcaactgggtgcgccaggcccctggacagggcctggaatggatgggctggatctacttcgccagcggcaactccgagtacaaccagaaattcaccggcagagtgaccatgacccgggacaccagcatcaacaccgcctacatggaactgagcagcctgaccagcgaggataccgccgtgtacttctgcgccagcctgtacgactacgactggtacttcgacgtgtggggccagggcacaatggtcaccgtgtctagc

SEQ ID NO: 4 - DNA, 45 bp, Artificial Sequence
  Nucleotide sequence of the (4GS)3 linker
ggaggcggaggctccggagggggaggatctgggggaggcggaagc

SEQ ID NO: 5 - DNA, 336 bp, Artificial Sequence
  Nucleotide sequence of the BCMA single chain variable fragment VL
gatatcgtgatgacccagacccccctgagcctgagcgtgacacctggacagcctgccagcatcagctgcaagagcagccagagcctggtgcacagcaacggcaacacctacctgcactggtatctgcagaagcccggccagagcccccagctgctgatctacaaggtgtccaaccggttcagcggcgtgcccgacagattttctggcagcggctccggcaccgacttcaccctgaagatctcccgggtggaagccgaggacgtgggcatctactactgcagccagtccagcatctacccctggaccttcggccaggggaccaagctggaaatcaaa

SEQ ID NO: 6 - DNA, 687 bp, Artificial Sequence
  Nucleotide sequence of the IgG4-Fc Hinge-CH2- CH3 4/2NQ
aaagagtctaagtacggaccgccttgtcctccttgtccagctcctcctgtggccggacctagcgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttccagagcacctaccgggtggtgtccgtgctgacagtgctgcaccaggactggctgaacggcaaagagtacaagtgcaaggtgtccaacaagggcctgcccagcagcatcgagaaaaccatcagcaaggccaagggccagcctcgcgagccccaggtgtacacactgcctccaagccaggaagagatgaccaagaaccaggtgtccctgacctgtctcgtgaagggcttctaccccagcgacattgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgagcctgggcaag

SEQ ID NO: 7 - DNA, 84 bp, Artificial Sequence
  Nucleotide sequence of the CD28 transmembrane domain
atgttctgggtgctggtggtcgtgggcggagtgctggcctgttacagcctgctcgtgaccgtggccttcatcatcttttgggtc

SEQ ID NO: 8 - DNA, 126 bp, Artificial Sequence
  Nucleotide sequence of the 4-1BB cytoplasmic domain
aagcggggcagaaagaagctgctgtatatcttcaagcagcccttcatgcggcccgtgcagaccacacaggaagaggacggctgctcctgccggttccccgaggaagaagaaggcggctgcgagctg

SEQ ID NO: 9 - DNA, 336 bp, Artificial Sequence
  Nucleotide sequence of the CD3 zeta domain
agagtgaagttcagcagaagcgccgacgcccctgcctatcagcagggccagaaccagctgtacaacgagctgaacctgggcagacgggaagagtacgacgtgctggataagcggagaggccgggaccctgagatgggcggcaagcctagaagaaagaacccccaggaaggcctgtataacgaactgcagaaagacaagatggccgaggcctacagcgagatcggaatgaagggcgagcggagaagaggcaagggccacgatggcctgtaccagggactgagcaccgccaccaaggatacctatgacgcactgcacatgcaggccctgccccccaga

SEQ ID NO: 10 - DNA, 72 bp, Artificial Sequence
  Nucleotide sequence of the T2A ribosomal skip element
ctcgagggcggaggcgaaggcagaggatctctgctgacatgcggcgacgtggaagagaaccctggccccaga

SEQ ID NO: 11 - DNA, 66 bp, Artificial Sequence
  Nucleotide sequence of the GMCSF signal peptide
atgctgctgctcgtgacaagcctgctgctgtgcgagctgccccaccctgcctttctgctgatcccc

SEQ ID NO: 12 - DNA, 1008 bp, Artificial Sequence
  Nucleotide sequence of the tEGFR sequence
cggaaagtgtgcaacggcatcggcatcggagagttcaaggacagcctgtccatcaacgccaccaacatcaagcacttcaagaattgcaccagcatcagcggcgacctgcacatcctgccagtggcctttagaggcgacagcttcacccacacccccccactggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 13 - protein, 1060 aa, Artificial Sequence
  Whole amino acid sequence of the chimeric antigen receptor (CAR) capable of binding to BCMA used in the Examples
MLLLVTSLLLCELPHPAFLLIPQVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKKESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 14 - protein, 22 aa, Artificial Sequence
  Amino acid sequence of the GMCSF signal peptide
MLLLVTSLLLCELPHPAFLLIP

SEQ ID NO: 15 - protein, 119 aa, Artificial Sequence
  Amino acid sequence of the BCMA single chain variable fragment VH
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSS

SEQ ID NO: 16 - protein, 15 aa, Artificial Sequence
  Amino acid sequence of the (4GS)3 linker
GGGGSGGGGSGGGGS

SEQ ID NO: 17 - protein, 112 aa, Artificial Sequence
  Amino acid sequence of the BCMA single chain variable fragment VL
DIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 18 - protein, 229 aa, Artificial Sequence
  Amino acid sequence of the IgG4-Fc Hinge-CH2- CH3 4/2NQ
KESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 28 aa, Artificial Sequence
  Amino acid sequence of the CD28 transmembrane domain
MFWVLVVVGGVLACYSLLVTVAFIIFWV

SEQ ID NO: 20 - protein, 42 aa, Artificial Sequence
  Amino acid sequence of the 4-1BB cytoplasmic domain
KRGRKKLLYIFKQPFMRPVQTTQEEDGCSCRFPEEEEGGCEL

SEQ ID NO: 21 - protein, 112 aa, Artificial Sequence
  Amino acid sequence of the CD3 zeta domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 24 aa, Artificial Sequence
  Amino acid sequence of the T2A ribosomal skip element
LEGGGEGRGSLLTCGDVEENPGPR

SEQ ID NO: 23 - protein, 22 aa, Artificial Sequence
  Amino acid sequence of the GMCSF signal peptide
MLLLVTSLLLCELPHPAFLLIP

SEQ ID NO: 24 - protein, 335 aa, Artificial Sequence
  Amino acid sequence of the tEGFR sequence
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 25 - DNA, 23 bp, Artificial Sequence
  BCMA forward primer
tgttcttctaatactcctcctct

SEQ ID NO: 26 - DNA, 20 bp, Artificial Sequence
  BCMA reverse primer
aactcgtcctttaatggttc

SEQ ID NO: 27 - DNA, 20 bp, Artificial Sequence
  beta-actin forward primer
tccatcatgaagtgtgacgt

SEQ ID NO: 28 - DNA, 22 bp, Artificial Sequence
  beta-actin reverse primer
gagcaatgatcttgatcttcat

(19) United States
(12) Patent Application Publication
(10) Pub. No.: US 2023/0346734 A1
(43) Pub. Date: Nov. 2, 2023

(54) COMBINATION THERAPY OF ATRA OR OTHER RETINOIDS WITH IMMUNOTHERAPEUTIC AGENTS BINDING TO BCMA

(71) Applicant: JULIUS-MAXIMILIANS-UNIVERSITÄT WÜRZBURG, Würzburg (DE)

(72) Inventors: Michael HUDECEK, Würzburg (DE); Estefania GARCÍA GUERRERO, Seville (ES); Sabrina PROMMERSBERGER, Rimpar (DE)

(21) Appl. No.: 17/914,062

(22) PCT Filed: Apr. 14, 2021
(86) PCT No.: PCT/EP2021/059657

§ 371 Date: Sep. 23, 2022

(30) Foreign Application Priority Data
Apr. 14, 2020 (EP) 20169313.2

(51) Int. Cl. A61K 31/203; A61K 45/06; A61K 47/68; A61P 35/00; A61K 39/395

(52) U.S. Cl. CPC A61K 31/203; A61K 45/06; A61K 47/6803; A61P 35/00; A61K 39/3955

(57) ABSTRACT

The invention relates to combination therapies of ATRA and other retinoids with immunotherapeutic agents binding to BCMA such as CAR-T cells capable of binding to BCMA, antibodies capable of binding to BCMA or antibody fragments capable of binding to BCMA. According to the invention, these combination therapies can be advantageously applied to the treatment of cancers such as multiple myeloma and can also be applied to the treatment of antibody-mediated autoimmune diseases. The combination therapies in the treatment of cancers according to the present invention are advantageous, for instance, because retinoids such as ATRA can upregulate BCMA mRNA levels as well as BCMA protein levels in cancer cells, such that the cancer cells can be more effectively targeted by immunotherapeutic anticancer agents capable of binding to BCMA such as CAR-T cells capable of binding to BCMA, antibodies capable of binding to BCMA or antibody fragments capable of binding to BCMA.

### FIELD OF THE INVENTION

The invention relates to combination therapies of ATRA and other retinoids with immunotherapeutic agents binding to BCMA such as CAR-T cells capable of binding to BCMA, antibodies capable of binding to BCMA or antibody fragments capable of binding to BCMA. According to the invention, these combination therapies can be advantageously applied to the treatment of cancers such as multiple myeloma and can also be applied to the treatment of antibody-mediated autoimmune diseases. The combination therapies in the treatment of cancers according to the present invention are advantageous, for instance, because retinoids such as ATRA can upregulate BCMA mRNA levels as well as BCMA protein levels in cancer cells, such that the cancer cells can be targeted more effectively by immunotherapeutic anticancer agents capable of binding to BCMA such as CAR-T cells capable of binding to BCMA, antibodies capable of binding to BCMA or antibody fragments capable of binding to BCMA. Additionally, ATRA and other retinoids can be combined with gamma-secretase inhibitors and BCMA-targeting immunotherapeutic agents, leading to an even further increased BCMA expression on the target cells and therefore, even better immunotherapeutic response.

### BACKGROUND OF THE INVENTION

Multiple myeloma (MM) is a largely incurable hematologic disease characterized by uncontrolled clonal proliferation of malignant plasma cells in the bone marrow^{1,2}. Despite recent approval of several new therapeutics myeloma is still considered of not being curable. The majority of patients becomes refractory or has to discontinue treatment due to toxicity and ultimately succumbs to the disease³⁻⁵.

Since CAR T-cells have been shown to induce durable complete remissions in other advanced hematologic malignancies like acute lymphocytic leukemia (ALL) and diffuse large B-cell lymphoma (DLBCL), significant efforts are underway to develop CAR-based therapies for MM⁶⁻¹⁰. Recently, B cell maturation antigen (BCMA) has increasingly drawn attention as a possible target antigen for MM treatment^{2, 11, 12}. BCMA is a tumor necrosis family receptor (TNFR) that is expressed by MM cells. It is also found on some healthy hematopoietic cells, such as plasma cells and plasmacytoid dendritic cells, but not on cells from healthy solid tissues. The favorable expression profile has fostered the development of a remarkable armamentarium of BCMA-specific immunotherapies including CAR T cell therapies¹³⁻¹⁹. In recent phase I/II clinical trials BCMA-CAR T-cells achieved partial and complete responses in fractions of MM patients^{16, 19}.

Retinoic acids can influence gene expression and protein production of cells²⁰. The use of all-trans retinoic acid (ATRA) has been widely investigated as treatment for some cancer types and it was shown, that it can induce major changes in post-translational modifications such as histone acetylation in tumor cells²¹⁻²⁴. Treatment with ATRA also induces epigenetic changes in MM cells, leading to enhanced expression of CD38 and subsequently enhanced efficacy of the CD38-targeting antibody daratumumab^{22, 25}.

Administration of gamma-secretase inhibitors (GSI) can also increase BCMA expression on MM cells, by blocking BCMA cleavage by the ubiquitous multi-subunit y-secretase complex, leading to improved MM cell recognition by BCMA-CAR-T cells⁴⁰.

Prior to the present invention, there remained a need in the art for more effective cancer therapies including therapies for multiple myeloma.

### DESCRIPTION OF THE INVENTION

The inventors have investigated if epigenetic changes induced by ATRA influence the BCMA surface expression and the release of soluble BCMA (BCMAs) molecules by cancer cells and in particular by MM cells. Furthermore, it was analyzed if these ATRA-induced changes also affect the efficacy of BCMA-CAR T-cells.

B cell maturation antigen (BCMA) is preferentially expressed by B lineage cells, including multiple myeloma (MM) cells. Due to its favorable expression pattern it represents a promising target for chimeric antigen receptor (CAR) therapy. Clinical trials with BCMA-CAR T-cells are currently running and achieved first encouraging results. However, there are several therapeutic limitations, such as low or non-uniform BCMA expression, as well as tumor relapse after antigen-loss or down-regulation. To overcome these hurdles, the inventors aimed to increase overall BCMA expression on cancer cells such as MM cells.

The inventors investigated the potential of all-trans retinoic acid (ATRA) to up-regulate BCMA on MM cells, thereby enhancing the performance of BCMA-specific CAR T-cells. By using quantitative RT-PCR and flow cytometry, it was observed that co-incubation with the retinoid ATRA can induce a significant increase of BCMA RNA levels and BCMA surface expression on primary MM cells and myeloma cell lines.

Importantly, BCMA-specific CAR T-cells showed enhanced recognition and lysis of target cell lines, when these were pretreated with ATRA. Cytokine release and proliferation of BCMA-CAR T-cells were enhanced after stimulation with ATRA-treated target cells in comparison to untreated target cells. Even in MM1.S/NSG mice, BCMA was up-regulated on the surface of tumor cells when the animals were injected with ATRA for several days. A combinatorial treatment with ATRA and BCMA-specific CAR T-cells led to a distinct and prolonged decline of tumor mass in comparison to single agent treatment.

In addition it was shown, that the effect of BCMA up-regulation on target cell lines can be further enhanced by combining ATRA with gamma secretase inhibitors (GSI). By combining the administration of both drugs, the efficiency of BCMA-CAR T-cells was increased even further in vitro and in vivo. Thus, the combined application of the two agents GSI and ATRA leads to an even greater effect regarding BCMA up-regulation and recognition by BCMA-CAR-T cells.

According to the invention, retinoids such as ATRA can be used to enhance BCMA-targeting immunotherapies, e.g., by increasing the BCMA baseline expression on tumor cells and by keeping it at a high level during the therapy.

Although the retinoid ATRA led to enhanced expression of BCMA on the surface of myeloma cells, no increase in shed soluble BCMA (sBCMA) was found in supernatants of ATRA-treated cells. This was an unexpected favorable effect of the retinoid ATRA, because 1) sBCMA is constantly found in the serum of myeloma patients and was thus expected to be increased upon treatment with ATRA, and because 2) an increase in sBCMA should be avoided because it may interfere with and inhibit the efficacy of BCMA-directed anticancer therapies.

Nevertheless, the inventors confirmed that the anti-MM reactivity of their BCMA CAR is not inhibited in the presence of high concentrations of sBCMA that may occur at a later time point of ATRA treatment and which is constantly found in the serum of myeloma patients.

According to the invention, the advantageous upregulation of BCMA can not only be achieved with ATRA but can also be achieved with other retinoids. These retinoids are considered to share the same more of action (e.g. as specific epigenetic modulators) and can therefore be used in accordance with the present invention.

The studies made by the inventors illustrate the advantageous effects of combining retinoids such as ATRA and immunotherapeutic agents capable of binding to BCMA such as BCMA-CAR T-cells for cancer treatment such as the treatment of myeloma.

Further, according to the invention, such combination therapies can also be advantageously applied to the treatment of antibody-mediated autoimmune diseases. Antibodies are secreted by B cells, mostly by plasma cells which are differentiated B cells. Autoantibodies are antibodies binding to the individual's own proteins and can induce autoimmune diseases (such as lupus erythematosus). Therefore, B cells and especially plasma cells can act as therapeutic targets for treatment of such autoimmune diseases. Several monoclonal antibodies against CD19, CD20 and CD22 have already been used to target multiple B cell subtypes. The CD20-targeting antibody Rituximab is already approved for use in rheumatoid arthritis, granulomatosis with polyangiitis and microscopic polyangiitis. [K Hofmann, et. al, Front. Immunol., 23 Apr. 2018, Targeting B Cells and Plasma Cells in Autoimmune Diseases, https://doi.org/10.3389/fimmu.2018.00835; A. Rubbert-Roth, et. al Efficacy and safety of various repeat treatment dosing regimens of rituximab in patients with active rheumatoid arthritis: results of a Phase III randomized study (MIRROR), Rheumatology, Volume 49, Issue 9, September 2010, Pages 1683-1693, https://doi.org/10.1093/rheumatology/keq116].

B cell maturation antigen (BCMA) is preferentially expressed by B lineage cells including plasma cells. Therefore, according to the invention, antibody-mediated autoimmune diseases can also be treated with the immunotherapeutic agents capable of binding to BCMA according to the invention. Here, administration of an upregulator of BCMA mRNA levels according to the invention, e.g. a retinoid according to the invention, is expected to enhance the efficiency of the treatment. Instead of immunotherapeutic agents capable of binding to BCMA according to the invention, immunotherapeutic agents comprising a gene therapy vector encoding a chimeric antigen receptor (CAR) capable of binding to BCMA, said gene therapy vector being a gene therapy vector for the in vivo expression of said CAR in immune cells, can also be used in accordance with the invention.

The present invention is exemplified by the following preferred embodiments:
1. An immunotherapeutic anticancer agent capable of binding to BCMA for use in a method of cancer immunotherapy against BCMA as cancer antigen in a human patient, wherein the method is a method wherein an upregulator of BCMA mRNA levels is to be administered to the human patient.
2. An upregulator of BCMA mRNA levels for use in a method of cancer immunotherapy against BCMA as cancer antigen in a human patient, wherein the method is a method wherein an immunotherapeutic anticancer agent capable of binding to BCMA is to be administered to the human patient.
3. A combination of an immunotherapeutic anticancer agent capable of binding to BCMA and an upregulator of BCMA mRNA levels for use in a method of cancer immunotherapy against BCMA as cancer antigen in a human patient.
4. A method of treating cancer by immunotherapy against BCMA as cancer antigen in a human patient, the method comprising administering an immunotherapeutic anticancer agent capable of binding to BCMA and an upregulator of BCMA mRNA levels to the human patient.
5. The immunotherapeutic anticancer agent for use of item 1, the upregulator for use of item 2, the combination for use of item 3, or the method of item 4, wherein the upregulator is a retinoid.
6. The immunotherapeutic anticancer agent for use, the upregulator for use, the combination for use, or the method, of any one of items 1 to 5, wherein the retinoid is a non-aromatic retinoid.
7. The immunotherapeutic anticancer agent for use, the upregulator for use, the combination for use, or the method, of item 6, wherein the non-aromatic retinoid is all-trans retinoic acid (ATRA), isotretionin (13-cis-retinoic acid), alitretinoin (9-cis-retinoic acid), retinal or retinol.
8. The immunotherapeutic anticancer agent for use, the upregulator for use, the combination for use, or the method, of any one of items 1 to 7, wherein the upregulator is all-trans retinoic acid (ATRA).
9. The immunotherapeutic anticancer agent for use, the upregulator for use, the combination for use, or the method, of item 5, wherein the retinoid is an aromatic retinoid.
10. The immunotherapeutic anticancer agent for use, the upregulator for use, the combination for use, or the method, of item 9, wherein the aromatic retinoid is a monoaromatic retinoid, preferably acitretin, etretinate or motretinid.
11. The immunotherapeutic anticancer agent for use, the upregulator for use, the combination for use, or the method, of item 9, wherein the aromatic retinoid is a polyaromatic retinoid, preferably adapalene, arotinoid, an acetylene retinoid such as tazarotene, or bexarotene.
12. The immunotherapeutic anticancer agent for use, the upregulator for use, the combination for use, or the method, of any one of items 1 to 11, wherein the cancer is a cancer susceptible to upregulation of BCMA mRNA levels by said upregulator.
13. The immunotherapeutic anticancer agent for use, the upregulator for use, the combination for use, or the method, of any one of items 1 to 12, wherein the cancer is a hematological cancer, preferably leukemia, lymphoma, or multiple myeloma.
14. The immunotherapeutic anticancer agent for use, the upregulator for use, the combination for use, or the method, of any one of items 1 to 13, wherein the cancer is a cancer in which some or all of the cancer cells express BCMA.
15. The immunotherapeutic anticancer agent for use, the upregulator for use, the combination for use, or the method, of any one of items 1 to 14, wherein the cancer is a multiple myeloma, a B-cell leukemia or a B-cell lymphoma.
16. The immunotherapeutic anticancer agent for use, the upregulator for use, the combination for use, or the method, of any one of items 1 to 15, wherein the cancer is a multiple myeloma.
17. The immunotherapeutic anticancer agent for use, the upregulator for use, the combination for use, or the method, of any one of items 1 to 16, wherein the immunotherapeutic anticancer agent capable of binding to BCMA comprises immune cells expressing a chimeric antigen receptor (CAR) capable of binding to BCMA.
18. The immunotherapeutic anticancer agent for use, the upregulator for use, the combination for use, or the method, of item 17, wherein the immune cells expressing the CAR capable of binding to BCMA are T cells expressing the CAR capable of binding to BCMA (CAR-T cells capable of binding to BCMA).
19. The immunotherapeutic anticancer agent for use, the upregulator for use, the combination for use, or the method, of any one of items 1 to 18, wherein the immunotherapeutic anticancer agent capable of binding to BCMA comprises an antibody capable of binding to BCMA or an antibody fragment capable of binding to BCMA, and wherein said antibody or antibody fragment is preferably a bispecific antibody which is more preferably selected from a BiTE or a DART.
20. The immunotherapeutic anticancer agent for use, the upregulator for use, the combination for use, or the method, of item 19, wherein antibody capable of binding to BCMA or antibody fragment capable of binding to BCMA is a conjugate with a drug.
21. The immunotherapeutic anticancer agent for use, the upregulator for use, the combination for use, or the method, of item 20, wherein the drug is an anticancer drug.
22. The immunotherapeutic anticancer agent for use, the upregulator for use, the combination for use, or the method, of any one of items 17 or 18, wherein the use leads to prolonged persistence of the immune cells and/or prolonged decline of tumor mass, compared to the cancer immunotherapy with the immune cells alone.
23. The immunotherapeutic anticancer agent for use, the upregulator for use, the combination for use, or the method, of any one of items 1 to 22, wherein the cancer is relapsed and refractory multiple myeloma or newly diagnosed multiple myeloma.
24. The immunotherapeutic anticancer agent for use, the upregulator for use, the combination for use, or the method, of any one of items 1 to 23, wherein in the method, a gamma secretase inhibitor is to be administered.
25. The immunotherapeutic anticancer agent for use, the upregulator for use, the combination for use, or the method, of item 24, wherein the gamma secretase inhibitor is semagacestat (LY 450139), crenigacestat (LY3039478), RO4929097, DAPT or MK-0752.
26. An immunotherapeutic agent capable of binding to BCMA for use in a method of treating an antibody-mediated autoimmune disease in a human patient, wherein the method is a method wherein an upregulator of BCMA mRNA levels is to be administered to the human patient.
27. An upregulator of BCMA mRNA levels for use in a method of treating an antibody-mediated autoimmune disease in a human patient, wherein the method is a method wherein an immunotherapeutic agent capable of binding to BCMA is to be administered to the human patient.
28. A combination of an immunotherapeutic agent capable of binding to BCMA and an upregulator of BCMA mRNA levels for use in a method of treating an antibody-mediated autoimmune disease in a human patient.
29. A method of treating an antibody-mediated autoimmune disease in a human patient, the method comprising administering an immunotherapeutic agent capable of binding to BCMA and an upregulator of BCMA mRNA levels to the human patient.
30. The immunotherapeutic agent for use of item 26, the upregulator for use of item 27, the combination for use of item 28, or the method of item 29, wherein the upregulator is as defined in any one of items 5-11.
31. The immunotherapeutic agent for use, the upregulator for use, the combination for use, or the method, of any one of items 26 to 30, wherein the immunotherapeutic agent capable of binding to BCMA comprises immune cells expressing a chimeric antigen receptor (CAR) capable of binding to BCMA.
32. The immunotherapeutic agent for use, the upregulator for use, the combination for use, or the method, of item 31, wherein the immune cells expressing the CAR capable of binding to BCMA are T cells expressing the CAR capable of binding to BCMA (CAR-T cells capable of binding to BCMA).
33. The immunotherapeutic agent for use, the upregulator for use, the combination for use, or the method, of any one of items 26 to 32, wherein the immunotherapeutic agent capable of binding to BCMA comprises an antibody capable of binding to BCMA or an antibody fragment capable of binding to BCMA.
34. The immunotherapeutic agent for use, the upregulator for use, the combination for use, or the method, of item 33, wherein antibody capable of binding to BCMA or antibody fragment capable of binding to BCMA is a conjugate with a drug.
35. The immunotherapeutic agent for use, the upregulator for use, the combination for use, or the method, of item 34, wherein the drug is a cytotoxic drug.
36. The immunotherapeutic agent for use, the upregulator for use, the combination for use, or the method, of any one of items 26-35, wherein the antibody-mediated autoimmune disease is Graves' disease, myasthenia gravis, lupus erythematosus, rheumatoid arthritis, goodpasture syndrome, scleroderma, CREST syndrome, granulomatosis with polyangiitis, microscopic polyangiitis, pemphigus vulgaris, Sjögren's syndrome, diabetes mellitus type 1, primary biliary cholangitis, Hashimoto's thyreoiditis, neuromyelitis optica spectrum disorders, anti-NMDA receptor encephalitis, vasculitis or multiple sclerosis.
37. An immunotherapeutic anticancer agent comprising a gene therapy vector encoding a chimeric antigen receptor (CAR) capable of binding to BCMA, said gene therapy vector being a gene therapy vector for the in vivo expression of said CAR in immune cells, for use in a method of cancer immunotherapy against BCMA as cancer antigen in a human patient, wherein the method is a method wherein an upregulator of BCMA mRNA levels is to be administered to the human patient.
38. An upregulator of BCMA mRNA levels for use in a method of cancer immunotherapy against BCMA as cancer antigen in a human patient, wherein the method is a method wherein an immunotherapeutic anticancer agent is to be administered to the human patient, said immunotherapeutic anticancer agent comprising a gene therapy vector encoding a chimeric antigen receptor (CAR) capable of binding to BCMA, said gene therapy vector being a gene therapy vector for the in vivo expression of said CAR in immune cells.
39. A combination of an immunotherapeutic anticancer agent and an upregulator of BCMA mRNA levels for use in a method of cancer immunotherapy against BCMA as cancer antigen in a human patient, said immunotherapeutic anticancer agent comprising a gene therapy vector encoding a chimeric antigen receptor (CAR) capable of binding to BCMA, said gene therapy vector being a gene therapy vector for the in vivo expression of said CAR in immune cells.
40. A method of treating cancer by immunotherapy against BCMA as cancer antigen in a human patient, the method comprising administering an immunotherapeutic anticancer agent and an upregulator of BCMA mRNA levels to the human patient, said immunotherapeutic anticancer agent comprising a gene therapy vector encoding a chimeric antigen receptor (CAR) capable of binding to BCMA, said gene therapy vector being a gene therapy vector for the in vivo expression of said CAR in immune cells.
41. The immunotherapeutic anticancer agent for use, the upregulator for use, the combination for use, or the method, of any one of items 37-40, wherein the upregulator is as defined in any one of items 5-11.
42. The immunotherapeutic anticancer agent for use, the upregulator for use, the combination for use, or the method, of any one of items 37-41, wherein the cancer is as defined in any one of items 12-16 or 23.
43. The immunotherapeutic anticancer agent for use, the upregulator for use, the combination for use, or the method, of any one of items 37-42, wherein in the method, a gamma secretase inhibitor is to be administered, and wherein the gamma secretase inhibitor is as defined in items 24 or 25.

### BRIEF DESCRIPTION OF THE DRAWINGS

FIG. 1. ATRA treatment leads to enhanced BCMA-expression on myeloma cell lines.

Flow cytometric analysis of BCMA-expression on MM.1S, OPM-2 and NCI-H929 cell lines that had been cultured in the absence or presence of 50 nM ATRA for 72 hours. Shaded histogram shows staining with anti-BCMA mAb, white histogram shows staining with isotype control antibody. 7-AAD was used to exclude dead cells from analysis. Inset number states the absolute difference in MFI of treated and non-treated cells to isotype.

FIG. 2. ATRA treatment leads to enhanced BCMA-expression on MM.1S, OPM-2 and NCI-H929 cells.

Bar diagrams show relative increase of BCMA expression on ATRA-treated myeloma cell lines normalized to untreated cells. Bar diagrams show mean values+SD (n=3). P-values between indicated groups were calculated using unpaired t-test. *p<0.05

FIG. 3. ATRA treatment leads to enhanced BCMA-expression on MM.1S cells.

Representative photographs of BCMA molecule distribution on untreated and ATRA-treated MM.1S cells visualized by direct stochastic optical reconstruction microscopy (dSTORM).

FIG. 4. BCMA upregulation by ATRA is reversible on myeloma cell lines.

The overlay histogram shows BCMA expression on untreated myeloma cell lines, 72 hours after ATRA treatment (50 nM), 24 hours after subsequent removal of the drug, and 72 hours after re-exposition to ATRA.

FIG. 5. ATRA treatment leads to enhanced BCMA-RNA levels in myeloma cell lines.

BCMA RNA levels in MM.1S (n=4) and OPM-2 (n=3) by quantitative reverse transcription PCR (qRT-PCR) assay was quantified after incubation with increasing doses of ATRA for 48 hours. Depicted are mean values+SD. P-values between indicated groups were calculated using unpaired t-test. *p<0.05.

FIG. 6. BCMA expression highly varies between myeloma patients.

Differential mean fluorescence intensity (MFI) of BCMA and isotype control staining is shown on CD3830 CD138+ myeloma cells from newly diagnosed (ND) and relapse/refractory (R/R) myeloma who had received previous treatment with immunomodulatory drugs and proteasome inhibitors (n=18). delta MFI is the differential MFI of BCMA and isotype control staining.

FIG. 7. ATRA treatment leads to enhanced BCMA-expression on primary myeloma cells.

Flow cytometric analysis of BCMA-expression on primary myeloma cells that had been cultured in the absence or presence of ATRA for 72 hours. 7-AAD was used to exclude dead cells from analysis.

FIG. 8. ATRA treatment leads to enhanced BCMA-expression on primary myeloma cells.

Bar diagram shows normalized BCMA expression on primary myeloma cells (n=5) before and after ATRA treatment. Depicted are mean values+SD. P-values between indicated groups were calculated using unpaired t-test. *p<0.05.

FIG. 9. BCMA up-regulation by ATRA is reversible on primary myeloma cells.

The overlay histogram shows BCMA expression on untreated primary myeloma cells 72 hours after ATRA treatment (100 nM), 24 hours after subsequent removal of the drug, and 72 hours after re-exposition to ATRA. 7-AAD was used to exclude dead cells from analysis.

FIG. 10. Combination of ATRA and GSI treatment leads to enhanced BCMA-expression on MM.1S and OPM-2 cells.

Bar diagram shows BCMA expression on MM.1S cells (n=5) and OPM-2 cells (n=3) after treatment with 100 nM ATRA and/or 0.01 μM GSI LY3039478 for 72 hours. Depicted are mean values+SD. P-values between indicated groups were calculated using unpaired t-test. *p<0.05

FIG. 11. ATRA treatment does not affect the viability of BCMA-CAR T-cells.

Viability of BCMA CD4+ and CD8+ CAR T-cells after incubation with increasing doses of ATRA for 72 hours determined by flow cytometry. The bar diagram shows the percentage of viable (7-AAD-) T cells after ATRA-treatment normalized to untreated cells. Data are presented as mean values+SD (n=3).

FIG. 12. ATRA treatment does not affect the CAR expression on BCMA-CAR T-cells.

EGFRt_BCMA-CAR transgene expression of BCMA CD4+ and CD8+ CAR T-cells after incubation with increasing doses of ATRA for 72 hours determined by flow cytometry. The bar diagram shows the percentage of EGFRt+ T cells after ATRA-treatment normalized to untreated cells. Data are presented as mean values+SD (n=3).

FIG. 13. BCMA-CAR T-cells confer enhanced cytotoxicity against ATRA or ATRA+GSI-treated MM.1S in vitro.

Myeloma cell lines were incubated with 100 nM ATRA and/or 0.01 uM GSI for 72 hours or were left untreated. Cytolytic activity of CD8⁺ BCMA-CAR T-cells was determined in a bioluminescence-based assay after 4 h of co-incubation with target cells. Assay was performed in triplicate wells with 5,000 target cells per well. Data are presented as mean values+SD of n=4 independent experiments. P-values between indicated groups were calculated using unpaired t-test. *p<0.05

FIG. 14. BCMA-CAR T-cells confer enhanced cytotoxicity against ATRA or ATRA+GSI-treated OPM-2 in vitro.

Myeloma cell lines were incubated with 100 nM ATRA and/or 0.01 uM GSI for 72 hours or were left untreated. Cytolytic activity of CD8⁺ BCMA-CAR T-cells was determined in a bioluminescence-based assay after 4 h of co-incubation with target cells. Assay was performed in triplicate wells with 5,000 target cells per well. Data are presented as mean values+SD of n=4 independent experiments. P-values between indicated groups were calculated using unpaired t-test. *p<0.05

FIG. 15. BCMA-CAR T-cells confer enhanced proliferative reactivity after stimulation with ATRA or ATRA+GSI-treated MM.1S in vitro.

MM.1S were incubated with 100 nM ATRA and/or 0.01 uM GSI for 72 hours or were left untreated. Afterwards, CFSE-labeled BCMA-CAR T-cells were co-incubated with these target cells. Proliferative capacity of BCMA-CAR T-cells was determined after three days by measuring the reduction of CFSE-labeling in effector cells. Data are presented as mean values+SD of n=3 independent experiments. P-values between indicated groups were calculated using unpaired t-test. *p<0.05

FIG. 16. BCMA-CAR T-cells confer enhanced cytokine release after stimulation with ATRA or ATRA+GSI-treated MM.1S in vitro.

MM.1S were incubated with 100 nM ATRA and/or 0.01 uM GSI for 72 hours or were left untreated. Afterwards, BCMA-CAR T-cells were co-incubated with these target cells for 20 hours. Cytokine release of BCMA-CAR T-cells was determined in the supernatant by ELISA. Assay was performed in triplicate wells. Data are presented as mean values+SD of n=3 independent experiments. P-values between indicated groups were calculated using unpaired t-test. *p<0.05

FIG. 17. ATRA enhances BCMA expression on MM.1S in vivo.

NSG mice were inoculated with MM.1S cells. After twelve days, mice were i.p. injected with 30 mg/kg ATRA for 4 days. BCMA-expression on MM.1S cells obtained from bone marrow of untreated and ATRA-treated mice was analysed by flow cytometry.

FIG. 18. Combinatorial treatments of ATRA and BCMA-CAR T-cells or ATRA, GSI and BCMA-CAR T-cells lead to enhanced eradication of MM.1S in vivo.

NSG mice were inoculated with 2×10 6 MM.1S cells (ffluc+GFP+). 14 days later, they were treated with 1×10⁶ BCMA-CAR T-cells (CD4+:CD8+ ratio=1:1). BCMA-CAR T-cells were given alone or in combination with ATRA (30 mg/kg body weight as i.p. injection), GSI LY3039478 (1 mg/kg body weight as i.p. injection) or both drugs. 12 doses of ATRA were injected between day 12 and day 27 (Monday-Friday). GSI was given within the same time span and mice received a total of 7 doses (each Monday, Wednesday and Friday). The average radiance of MM.1S signal was analyzed to assess myeloma progression/regression in each treatment group. Bioluminescence (BMI) values were obtained as photon/sec/cm2/sr in regions of interest encompassing the entire body of each mouse. A) Time course of the experiment. The grey box marks the time period in which GSI and ATRA were administered. B) Graphs show the percentage change of bioluminescence signal from baseline values derived on day 14. Each bar represents mean value per mouse group. n=3-6 mice per group

FIG. 19. ATRA does not increase sBCMA in cell line supernatants.

Soluble BCMA concentration in the supernatant of MM.1S and OPM-2 cells after incubation with increasing doses of ATRA. Cell lines were cultured at 1×10⁶/well for 24 hours. After incubation, supernatant was collected and analyzed by ELISA. The stimulation was performed in triplicates. Depicted are mean values+SD

FIG. 20. sBCMA levels in the serum of myeloma patients increase with tumor burden.

Soluble BCMA concentration in the serum of MM patients. Peripheral blood from MM patients was collected. Centrifugation at 3,000 rpm for 10 min was performed to obtain the serum which was analyzed by ELISA (stimulation performed in triplicates). PD, progressive disease; SD, stable disease; PR, partial remission; CR, complete remission.

FIG. 21. Soluble BCMA is not abrogating the effect of BCMA-CAR T-cells against ATRA-treated myeloma cells.

CD8+ BCMA-CAR T-cells were co-cultured with MM.1S or K562/BCMA target cells in absence or presence of 150 ng/ml of soluble BCMA. After 4 hours, luciferin was added to the culture and the cytotoxicity was evaluated with a bioluminescence-based assay. Data show mean values of technical triplicates±SD.

### DEFINITIONS AND EMBODIMENTS

Unless otherwise defined below, the terms used in the present invention shall be understood in accordance with the common meaning known to the person skilled in the art.

Each publication, patent application, patent, and other reference cited herein is incorporated by reference in its entirety for all purposes to the extent that it is not inconsistent with the present invention. References are indicated by their reference numbers and their corresponding reference details which are provided in the “references” section.

The terms “K_{D}” or “K_{D} value” relate to the equilibrium dissociation constant as known in the art. In the context of the present invention, these terms can relate to the equilibrium dissociation constant of an immunotherapeutic agent or anticancer agent capable of binding to BCMA (e.g. a CAR T-cell or an antibody) with respect to the antigen of interest (i.e. BCMA). The equilibrium dissociation constant is a measure of the propensity of a complex (e.g. an antigen-targeting agent complex) to reversibly dissociate into its components (e.g. the antigen and the targeting agent). Methods to determine K_{D} values are known in art.

The chimeric antigen receptor is capable of binding to one or more antigens, preferably cancer antigens, more preferably cancer cell surface antigens. In a preferred embodiment, the chimeric antigen receptor is capable of binding to the extracellular domain of a cancer antigen. In a particularly preferred embodiment, the chimeric antigen receptor is capable of binding to the extracellular domain of BCMA and is even more preferably a chimeric antigen receptor encoded by the nucleic acid sequence of SEQ ID NO: 1 and/or a chimeric antigen receptor having the amino acid sequence of SEQ ID NO: 13.

In accordance with the invention, immune cells such as T cells, NK cells or PBMCs can be isolated from a patient, genetically modified (e.g. transduced) with a gene transfer vector encoding a chimeric antigen receptor according to the invention and administered to the patient in accordance with the methods and uses of the invention. In a preferred embodiment, the T cells are CD8⁺ T cells or CD4⁺ T cells. Alternatively, allogenic immune cells such as T cells, NK cells or PBMCs, from donors, preferably healthy donors, can be used. They can be genetically modified (e.g. transduced) with a gene transfer vector encoding a chimeric antigen receptor according to the invention and administered to the patient in accordance with the methods and uses of the invention. In a preferred embodiment, the T cells are CD8⁺ T cells or CD4⁺ T cells.

CAR NK cell therapy has been described, for instance, in [Liu E, et. al. Use of CAR-Transduced Natural Killer Cells in CD19-Positive Lymphoid Tumors. N Engl J Med. 2020 Feb. 6; 382(6):545-553. doi: 10.1056/NEJMoa1910607].

For CAR T cell therapy, T cells are usually manipulated and expanded ex vivo. However, in accordance with the invention, there is also the option to conduct gene transfer in vivo. One way to program immune cells such as T cells within the body is the gene transfer with DNA-carrying nanoparticles. This has, for instance, been described by Smith et al. [T. T. Smith, et. al, In situ programming of leukaemia-specific T cells using synthetic DNA nanocarriers, Nat Nanotechnol. 2017 August; 12(8): 813-820. Published online 2017 Apr. 17. doi: 10.1038/nnano.2017.57]. A second strategy is the in vivo CAR immune cell (e.g. CAR T cell) generation with viral vectors. This has, for instance, been described by Agarwal et al. [Agarwal S, et. al, Oncoimmunology. 2019 Oct. 10; 8(12):e1671761. In vivo generated human CAR T cells eradicate tumor cells. doi: 10.1080/2162402X.2019.1671761].

“Immune cells” as used in the invention are not particularly limited and include, for example, T cells, NK cells or PBMCs. In a preferred embodiment, the T cells are CD8⁺ T cells or CD4⁺ T cells.

The term “antibody” as used herein refers to any functional antibody that is capable of specific binding to the antigen of interest. Without particular limitation, the term antibody encompasses antibodies from any appropriate source species, including avian such as chicken and mammalian such as mouse, goat, non-human primate and human. Preferably, the antibody is a humanized or human antibody. Humanized antibodies are antibodies which contain human sequences and a minor portion of non-human sequences which confer binding specificity to an antigen of interest (e.g. BCMA). The antibody is preferably a monoclonal antibody which can be prepared by methods well-known in the art. The term antibody encompasses an IgG-1, -2, -3, or -4, IgE, IgA, IgM, or IgD isotype antibody. The term antibody encompasses monomeric antibodies (such as IgD, IgE, IgG) or oligomeric antibodies (such as IgA or IgM). The term antibody also encompasses—without particular limitations—isolated antibodies and modified antibodies such as genetically engineered antibodies, e.g. chimeric antibodies or bispecific antibodies, or antibody conjugates with a drug such as an anticancer drug or a cytotoxic drug. A preferred bispecific antibody capable of binding to BCMA in accordance with the invention can be a T-cell engager such as a BiTE (Bi-specific T-cell engager), e.g. a CD3xBCMA BiTE, or a DART (dual-affinity re-targeting proteins). An “antibody” (e.g. a monoclonal antibody) or “a fragment thereof” as described herein may have been derivatized or be linked to a different molecule. For example, molecules that may be linked to the antibody are other proteins (e.g. other antibodies), a molecular label (e.g. a fluorescent, luminescent, colored or radioactive molecule), a pharmaceutical and/or a toxic agent. The antibody or antigen-binding portion may be linked directly (e.g. in form of a fusion between two proteins), or via a linker molecule (e.g. any suitable type of chemical linker known in the art).

An antibody fragment or fragment of an antibody capable of binding to BCMA as used herein refers to a portion of an antibody that retains the capability of the antibody to specifically bind to the BCMA antigen. This capability can, for instance, be determined by determining the capability of the antigen-binding portion to compete with the antibody for specific binding to the antigen by methods known in the art. Without particular limitation, the antibody fragment can be produced by any suitable method known in the art, including recombinant DNA methods and preparation by chemical or enzymatic fragmentation of antibodies. Antibody fragments may be Fab fragments, F(ab′) fragments, F(ab′)2 fragments, single chain antibodies (scFv), single-domain antibodies, diabodies or any other portion(s) of the antibody that retain the capability of the antibody to specifically bind to the antigen.

Terms such as “treatment of cancer” or “treating cancer” or “cancer therapy” or “cancer immunotherapy” according to the present invention refer to a therapeutic treatment. An assessment of whether or not a therapeutic treatment works can, for instance, be made by assessing whether the treatment inhibits cancer growth in the treated patient or patients. Preferably, the inhibition is statistically significant as assessed by appropriate statistical tests which are known in the art. Inhibition of cancer growth may be assessed by comparing cancer growth in a group of patients treated in accordance with the present invention to a control group of untreated patients, or by comparing a group of patients that receive a standard cancer treatment of the art plus a treatment according to the invention with a control group of patients that only receive a standard cancer treatment of the art. Such studies for assessing the inhibition of cancer growth are designed in accordance with accepted standards for clinical studies, e.g. double-blinded, randomized studies with sufficient statistical power. The term “treating cancer” includes an inhibition of cancer growth where the cancer growth is inhibited partially (i.e. where the cancer growth in the patient is delayed compared to the control group of patients), an inhibition where the cancer growth is inhibited completely (i.e. where the cancer growth in the patient is stopped), and an inhibition where cancer growth is reversed (i.e. the cancer shrinks). An assessment of whether or not a therapeutic treatment works can be made based on known clinical indicators of cancer progression. In the context of cancers which do not form solid tumors, cancer growth may be assessed by known methods such as methods based on a counting of the cancer cells.

A “treatment of cancer” or “treating cancer” or “cancer therapy” or “cancer immunotherapy” as used in accordance with the present invention is preferably a treatment of the cancer itself. Alternatively, a “treatment of cancer” or “treating cancer” or “cancer therapy” or “cancer immunotherapy” in accordance with the invention can be a treatment of a precancerous condition which is preferably selected from multiple myeloma precursor states such as MGUS (Monoclonal Gammopathy of Undetermined Significance) and smoldering multiple myeloma.

A treatment of cancer according to the present invention does not exclude that additional or secondary therapeutic benefits also occur in patients, such as a treatment of amyloidosis, e.g. an amyloidosis associated with multiple myeloma.

The treatment of cancer according to the invention can be a first-line therapy, a second-line therapy, a third-line therapy, or a fourth-line therapy. The treatment can also be a therapy that is beyond fourth-line therapy. The meaning of these terms is known in the art and in accordance with the terminology that is commonly used by the US National Cancer Institute.

The method in accordance with the invention such as the method of cancer immunotherapy or the method of treating cancer by immunotherapy may, in one embodiment, be a method wherein in the method, an epigenetic modulator is also to be administered. Epigenetic modulators in accordance with the invention can be BET inhibitors, histone acetyltransferase inhibitors, histone deacetylase inhibitors, or DNA methyltransferase inhibitors and are preferably selected from the group consisting of valproic acid, butyric acid, panobinostat lactate, belinostat, vorinostat, dacinostat, entinostat, mocetinostat, romidepsin, and ricolinostat.

The term “capable of binding” as used herein refers to the capability to form a complex with a molecule that is to be bound (e.g. BCMA). Binding typically occurs non-covalently by intermolecular forces, such as ionic bonds, hydrogen bonds and Van der Waals forces and is typically reversible. Various methods and assays to determine binding capability are known in the art. Binding is usually a binding with high affinity, wherein the affinity as measured in K_{D} values is preferably is less than 1 μM, more preferably less than 100 nM, even more preferably less than 10 nM, even more preferably less than 1 nM, even more preferably less than 100 pM, even more preferably less than 10 pM, even more preferably less than 1 pM.

As used herein, each occurrence of terms such as “comprising” or “comprises” may optionally be substituted with “consisting of” or “consists of”.

A “combination” according to the invention is not limited to a particular mode of administration. The immunotherapeutic agent or anticancer agent capable of binding to BCMA and the upregulator of BCMA mRNA levels can, for example, be administered separately but at the same time, or in one composition and at the same time, or they can be administered separately and at separate time points.

Whether a substance is an upregulator of BCMA mRNA levels can be determined by methods known in the art, e.g. by measuring BCMA mRNA levels in the cells of interest, e.g. in the cancer cells, by methods such as quantitative RT-PCR, e.g. as described herein in the section “Quantitation of BCMA mRNA levels”.

Compositions and formulations in accordance with the present invention, which contain the immunotherapeutic anticancer agent capable of binding to BCMA and/or the upregulator of BCMA mRNA levels, are prepared in accordance with known standards for the preparation of pharmaceutical compositions and formulations. For instance, the compositions and formulations are prepared in a way that they can be stored and administered appropriately, e.g. by using pharmaceutically acceptable components such as carriers, excipients or stabilizers. Such pharmaceutically acceptable components are not toxic in the amounts used when administering the pharmaceutical composition or formulation to a patient. The pharmaceutical acceptable components added to the pharmaceutical compositions or formulations can be selected based on the chemical nature of the active agents (e.g. the immunotherapeutic anticancer agent capable of binding to BCMA and/or the upregulator of BCMA mRNA levels), the particular intended use of the pharmaceutical compositions and the route of administration. It is understood that in accordance with the invention, the compositions or formulations are suitable for administration to humans.

A pharmaceutically acceptable carrier, including any suitable diluent or, can be used herein as known in the art. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. Pharmaceutically acceptable carriers include, but are not limited to, saline, buffered saline, dextrose, water, glycerol, sterile isotonic aqueous buffer, and combinations thereof. It will be understood that the formulation will be appropriately adapted to suit the mode of administration.

### EXAMPLES

The present invention is exemplified by the following non-limiting examples.

The materials and methods used in the present examples were as follows:

### Human Subjects

Peripheral blood and bone marrow samples were obtained from healthy donors and myeloma patients after written informed consent to participate in research protocols approved by the Institutional Review Boards of the University of Wurzburg.

### Cell Lines

The K562, OPM-2, NCI-H929 and MM.1S cell lines were obtained from the German Collection of Microorganisms and Cell Cultures (DSMZ, Braunschweig, Germany). K562, OPM-2 and MM.1S cell lines were modified with firefly-luciferase_GFP by lentiviral transduction. K562 expressing full-length human BCMA was generated by transducing the K562-ffluc cell line with a BCMA-encoding lentiviral vector.

### Flow Cytometry

Bone marrow mononuclear cells (BMMC) were stained with anti-CD38 and anti-CD138 mAbs (Biolegend, Koblenz, Germany) to identify malignant plasma cells and anti-BCMA mAb (BioLegend; Clone: 19F2) or isotype control (Biolegend; mouse IgG2a,κ) according to the manufacturers instructions. Flow cytometry was done on a Canto II (BD, Heidelberg, Germany) and data analyzed using FlowJo software (TreeStar, Ashland, OR).

### ATRA-Treatment of Myeloma Cells

Myeloma cells were cultured in RPMI-1640 (Gibco, Darmstadt, Germany) supplemented with 10% fetal bovine serum at 1×10⁶ cells/ml. ATRA (Sigma-Aldrich, Darmstadt, Germany) was reconstituted in dimethyl sulfoxide and added to the medium to a final concentration of 25, 50 or 100 nM.

### In Vitro T-Cell Functional Assays

Cytolytic activity was analyzed in a bioluminescence-based assay using firefly-luciferase (ffluc)-transduced target cells. Proliferation was measured by dilution of CFSE proliferation dye by flow cytometry. Therefore, CFSE-labeled CAR T-cells were incubated with target cells for 72 h at a 4:1 effector to target cell ratio. IFNγ and IL-2 were measured by ELISA (Biolegend, Koblenz, Germany) in supernatants obtained after a 20 hour co-culture of T-cells with target cells (effector:target ratio=4:1).

### Quantitation of BCMA mRNA Levels

Total RNAs were extracted with RNeasy Mini Kit (Qiagen, Hilden, Germany) according to the manufacturer's protocol. Reverse transcription-quantitative polymerase chain reaction (RT-qPCR) analysis of BCMA was performed with 1 μg of total RNA and SuperScript™ II Reverse Transcriptase (Thermo Fisher Scientific, Inc Massachusetts). The quality and integrity of the RNA was verified by a Bioanalyzer 2100 (Agilent Technologies, Santa Clara, CA). The primer sequences used were as follows: BCMA forward primer, 5′-TGT TCT TCT AAT ACT CCT CCT CT-3′ (SEQ ID NO: 25) and reverse primer, 5′-AAC TCG TCC TTT AAT GGT TC-3′ (SEQ ID NO: 26). Primers specific for β-actin were used as a control (forward, 5′-TCC ATC ATG AAG TGT GAC GT-3′ (SEQ ID NO: 27) and reverse, 5′-GAG CAA TGATCTTGATCT TCA T-3′ (SEQ ID NO: 28)). RT-qPCR was performed in a 7900HT Real-time PCR System (Thermo Fisher Scientific, Inc Massachusetts) using Quantitec SYBR green Kit (Qiagen, Hilden, Germany) in a 7900 HT Fast Real Time PCR System (Applied Biosystems, Foster City, CA). PCR conditions consisted of the following: 95° C. for 3 min for denaturation; 95° C. for 30 sec for annealing; and 62° C. for 40 sec for extension, for 40 cycles. The threshold cycle for each sample was selected from the linear range and converted to a starting quantity by interpolation from a standard curve generated on the same plate for each set of primers. The BCMA messenger (m) RNA levels were normalized for each well to the β-actin mRNA levels using the 2-ΔΔCq method (21).

### ATRA Up-Regulates BCMA Expression on MM.1S In Vivo

The University of Wurzburg Institutional Animal Care and Use Committee approved all mouse experiments. Six- to eight-week old female NSG (NOD-scid IL2rynull) mice were obtained from Charles River and inoculated by tail vein injection with 2×10⁶ MM.1S/ffluc_GFP at day 0 and randomly allocated to ATRA-treatment and control group. ATRA (Sigma Aldrich, Darmstadt, Germany) was formulated in corn oil and administered by intraperitoneal (i.p) injection (30 mg/kg) for four days, starting twelve days after tumor inoculation. At the experiment end point on day 16, bone marrow samples of these mice were analyzed to study BCMA expression on MM.1S by a Canto II (BD, Heidelberg, Germany) and data analyzed using FlowJo software (TreeStar, Ashland, OR).

### In Vivo Experiment with Combined ATRA and GSI

To examine the combinatorial treatment with BCMA-CAR T-cells, ATRA and GSI female NSG (NOD-scid IL2rynull) mice were inoculated by tail vein injection with 2×10⁶ MM.1S/ffluc_GFP on day 0 and randomly allocated to treatment and control groups. On day 14, mice received a single dose of 1×10⁶ T-cells (i.e., 0.5×10⁶ CD4⁺ and 0.5×10⁶ CD8⁺) by tail vein injection. ATRA (Sigma Aldrich, Darmstadt, Germany) was diluted in DMSO, formulated in PEG300, Tween80 and saline and administered by intraperitoneal injection (i.p) at a dose of 30 mg/kg from Monday to Friday for 16 days, starting twelve days after tumor inoculation. GSI LY3039478 (Med Chem Express, NJ 08852, USA) was diluted in DMSO, formulated in PEG300, Tween80 and saline and administered by intraperitoneal injection (i.p) at a dose of 1 mg/kg Monday, Wednesday and Friday for 16 days, starting twelve days after tumor inoculation. Bioluminescence imaging was performed on an IVIS Lumina (Perkin Elmer, Waltham, MA) following i.p injection of D-luciferin (0.3 mg/g body weight) (Biosynth, Staad, Switzerland), and data was analyzed using Living Image software (Perkin Elmer).

### Statistical Analyses

Statistical analyses were performed using Prism software v6.07 (GraphPad, San Diego, California). Unpaired t-tests were used to analyze data obtained from in vitro and in vivo experiments. P-values<0.05 were considered statistically significant.

### Example 1: ATRA Augments BCMA Surface Expression on Myeloma Cell Lines

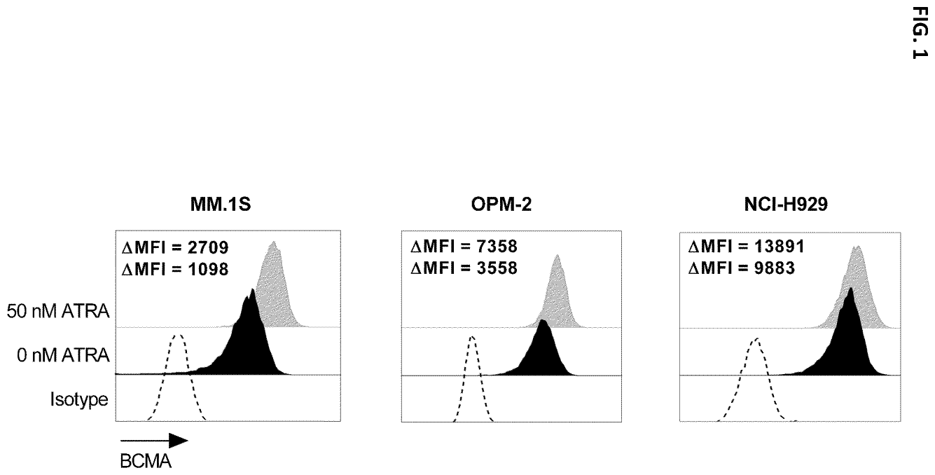

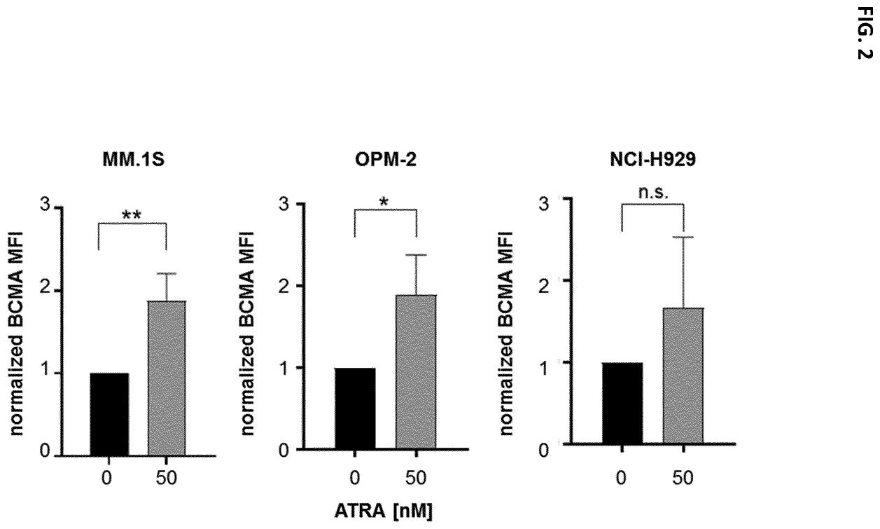

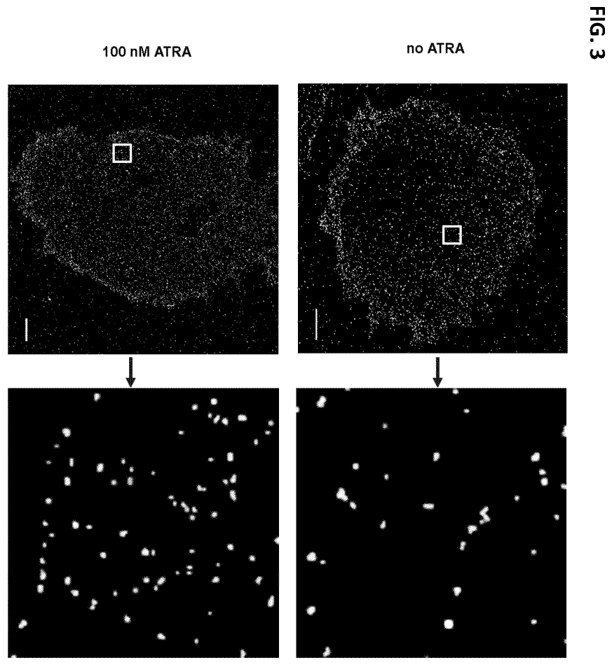

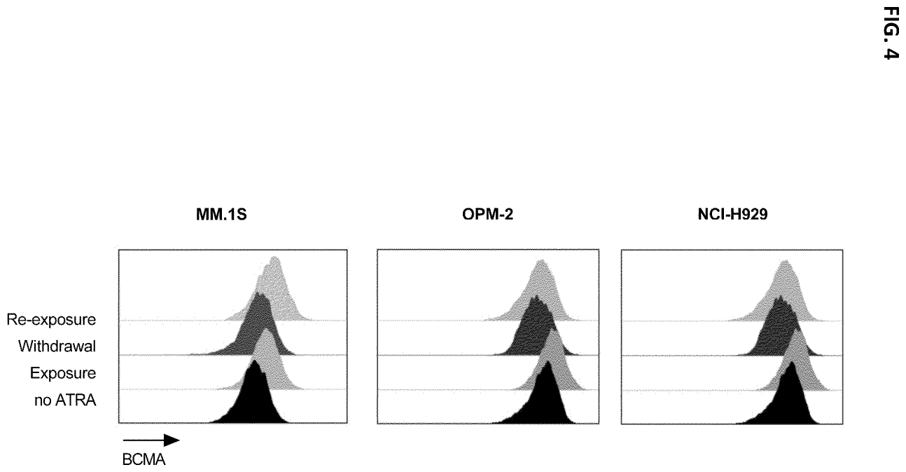

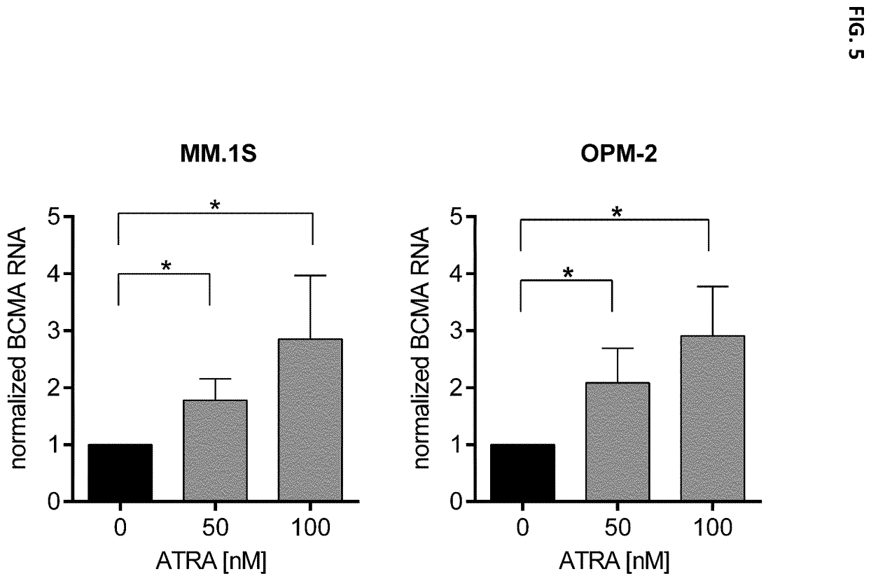

The inventors determined BCMA expression on three commonly utilized myeloma cell lines by flow cytometry and found graded BCMA expression with MM.1S being BCMA^{low} (deltaMFI: 1,098), OPM-2 being BCMA^{intermediate} (deltaMFI: 3,558) and NCI-H929 being BCMA^{high} (deltaMFI: 9,883) (FIG. 1). Then, the inventors treated each myeloma cell line with ATRA for 72 hours and re-examined BCMA expression by flow cytometry. The inventors found that BCMA expression had increased in all three myeloma cell lines, and that the hierarchy in BCMA expression had remained unchanged: MM.1S (deltaMFI: 2,709)<OPM-2 (deltaMFI: 7,358)<NCI-H929 (deltaMFI: 13,891) (FIG. 1). The inventors normalized the deltaMFI obtained at baseline to 1 and thus, the relative increase in BCMA expression after ATRA treatment was 1.9-fold in MM.1S (FIG. 2) and OPM-2 myeloma cells (FIG. 2), and 1.7-fold in NCI-H929 myeloma cells (FIG. 2). Upon discontinuation of ATRA treatment, BCMA expression returned to baseline levels within 72 hours in all three myeloma cell lines, but increased again with the same amplitude when ATRA treatment was recommenced (FIG. 4). The increase of BCMA surface molecules on MM.1S cells after ATRA treatment was additionally confirmed by single-molecule sensitive super-resolution microscopy using direct Stochastic Optical Reconstruction Microscopy dSTORM (FIG. 3). The inventors hypothesized that ATRA induces epigenetic changes in myeloma cells that lead to increased BCMA gene expression and confirmed by qPCR that this was indeed the case. On example of MM.1S and OPM-2, the relative increase in BCMA transcripts after 50 nM ATRA treatment was 1.8-fold and 2.1-fold, respectively (FIG. 5). Taken together, these data show that treatment with ATRA leads to increased expression of BCMA RNA and BCMA protein on the surface of MM.1S, OPM-2 and NCI-H929 myeloma cells.

### Example 2: ATRA Up-Regulates BCMA Surface Expression on Primary Myeloma Cells

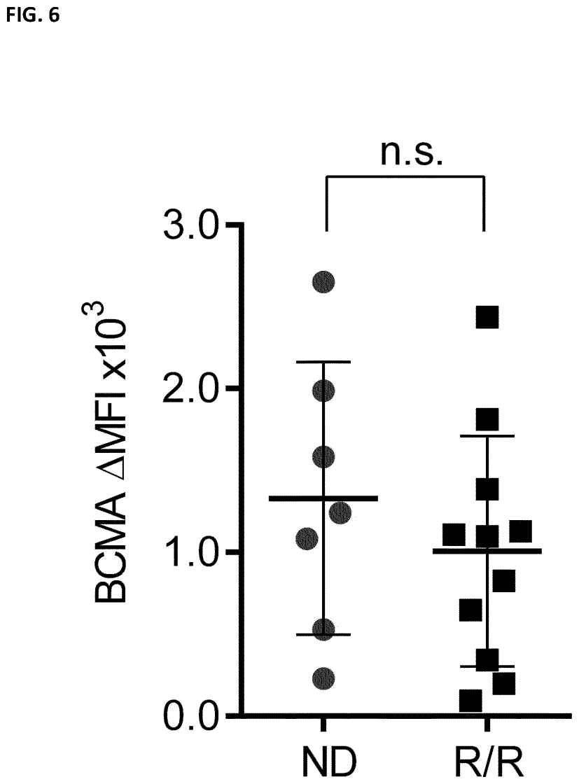

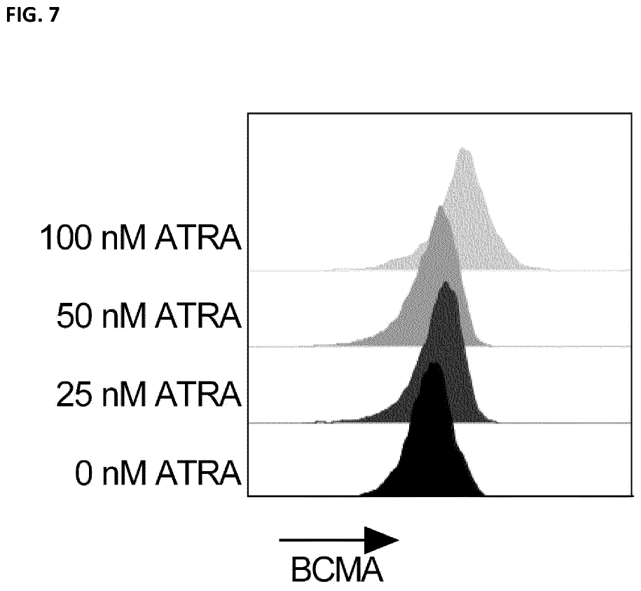

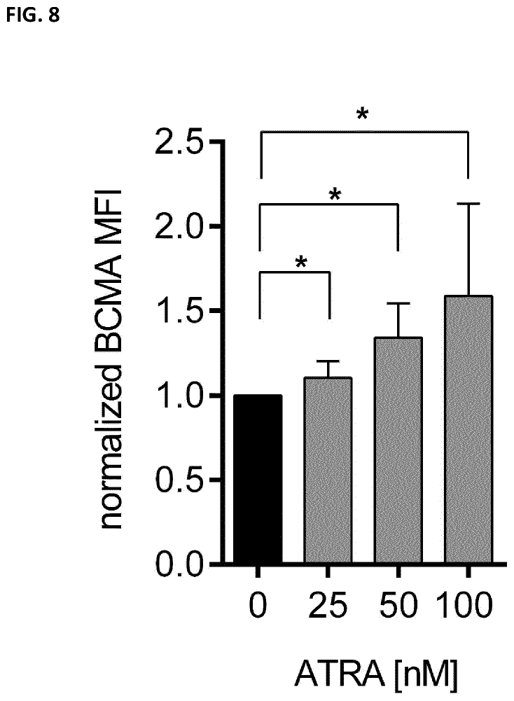

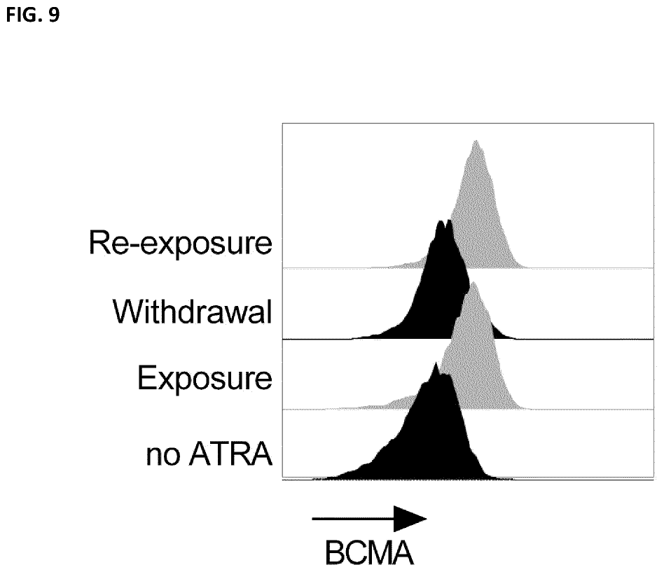

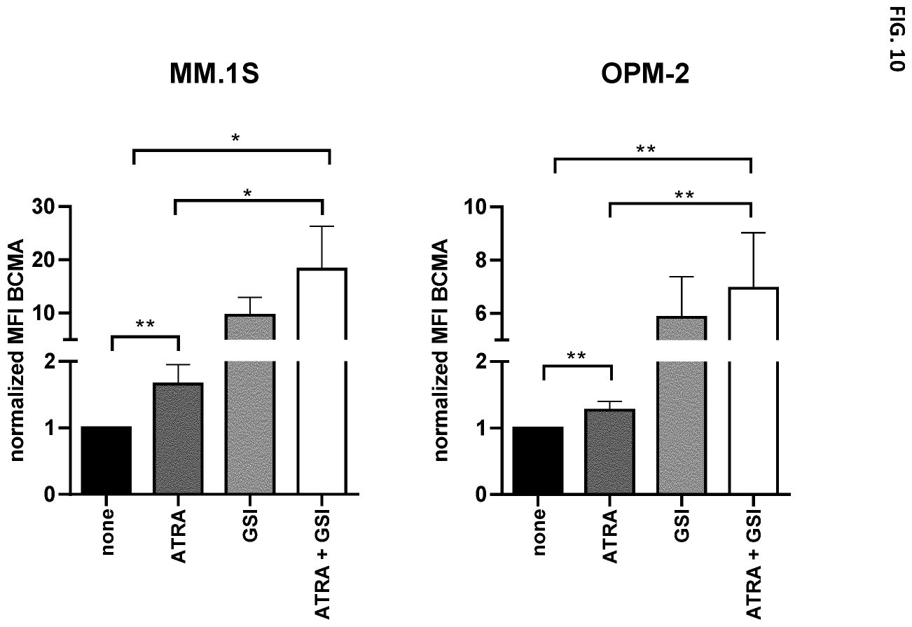

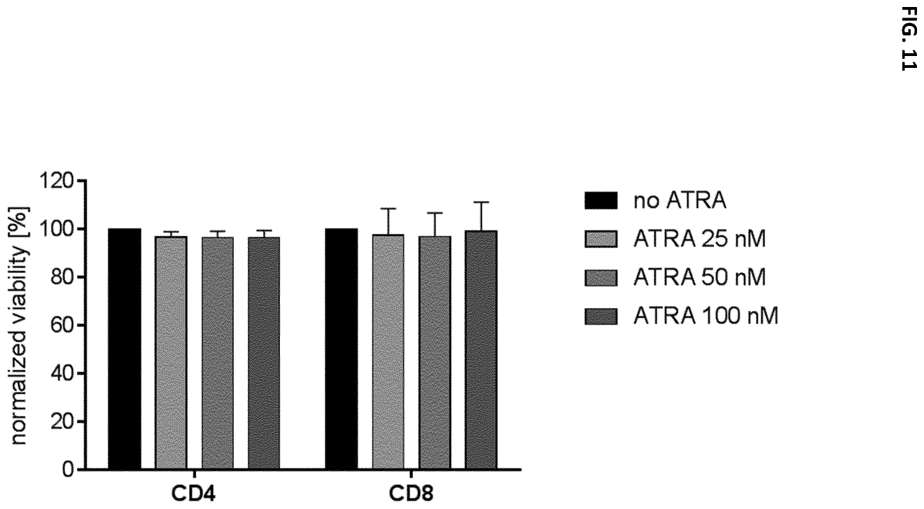

To corroborate their findings in primary myeloma cells, the inventors obtained bone marrow from patients with newly diagnosed (ND, n=7) and relapsed/refractory (R/R, n=11) myeloma. Patients in the R/R cohort had previously received treatment with immunomodulatory drugs and/or proteasome inhibitors, none of the patients had received anti-BCMA therapy. The inventors analyzed purified CD38⁺ CD138+malignant plasma cells by flow cytometry and found variable BCMA expression as assessed by deltaMFI between patients (deltaMFI^{low}=94; deltaMFI^{high}=2,650). There was no significant difference in BCMA expression on myeloma cells from ND and R/R patients (FIG. 6). From n=5 patients there was a sufficient number of primary myeloma cells to perform ATRA treatment and sequential analysis of BCMA expression (FIG. 7). Amongst these 5 patients, there were 3 ND and 2 R/R patients, and they evenly covered the spectrum of low to high BCMA expression determined above. In each of these 5 patient samples, the inventors detected a substantial increase in BCMA expression by flow cytometry after treatment with ATRA for 72 hours. A significant increase could be observed with ATRA used at all dose levels (100 nM P=0.04, 50 nM P=0.006 and 25 nM P=0.04) (FIG. 8). The increase in deltaMFI for BCMA expression that the inventors observed with primary myeloma cells after 100 nM ATRA treatment was on average 1.6× on average (1.23 fold-2.23 fold). Also with primary myeloma cells, BCMA expression declined to baseline levels once exposure to ATRA was discontinued, and increased again upon re-exposure to the drug (FIG. 9). Taken together, these data show that ATRA treatment augments BCMA surface expression on primary myeloma cells in patients with ND and R/R disease.

### Example 3: ATRA in Combination with GSI Further Increases BCMA Expression on Myeloma Cells Lines

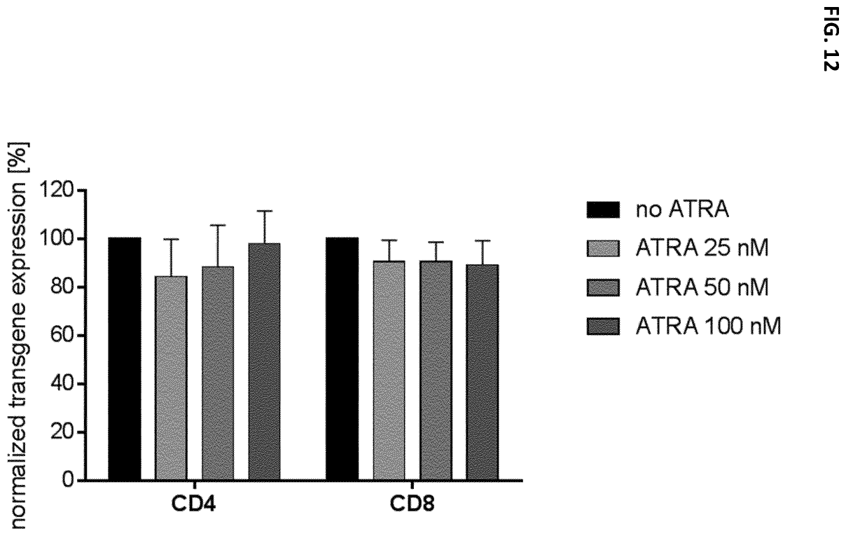

GSI can induce an increase in BCMA expression on myeloma cells, as they prevent shedding of BCMA molecules from the cell surface⁴⁰. The inventors determined whether the combination of ATRA and GSI can further increase BCMA expression on myeloma cells and if it can further improve the anti-myeloma reactivity of BCMA-CAR T-cells beyond the effect of ATRA alone. Treatment of MM.1S and OPM-2 cells with 100 nM ATRA and 0.01 μM GSI LY3039478 for 72 h led to a significant increase in BCMA expression. The combination of both drugs resulted in higher BCMA expression than the single use of one of the two drugs alone (FIG. 10).

### Example 4: BCMA-CAR T-Cells Confer Enhanced Reactivity Against ATRA-Treated Myeloma Cells

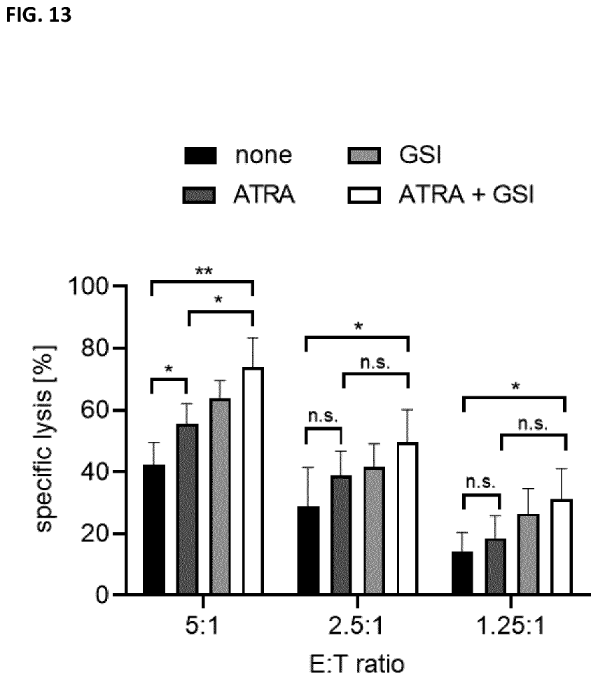

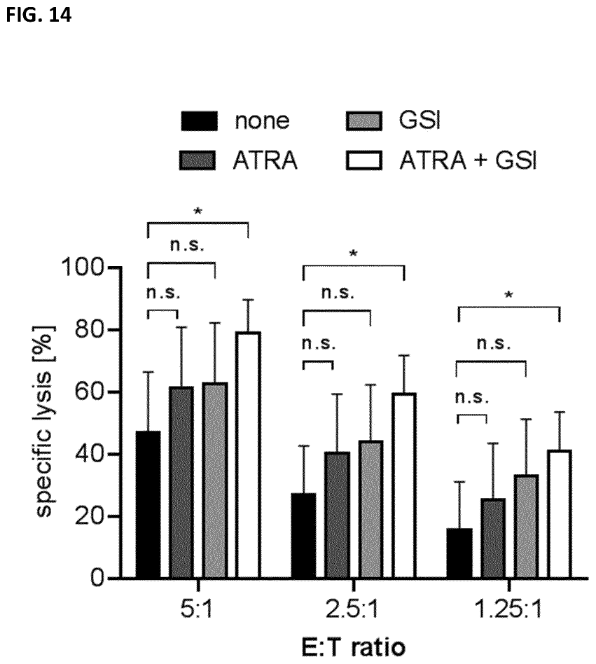

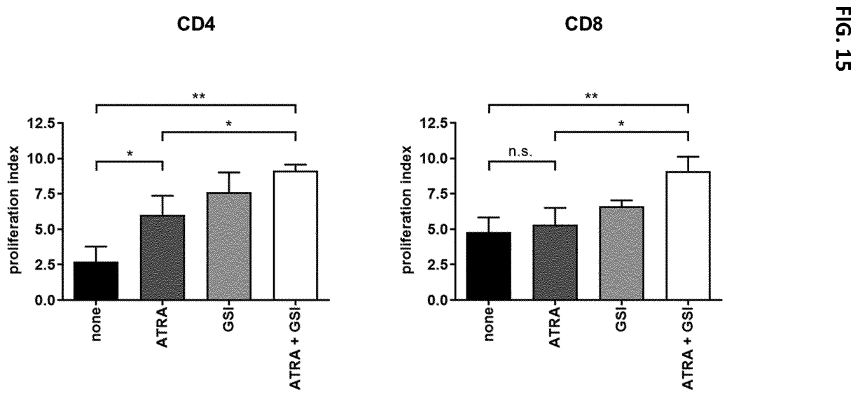

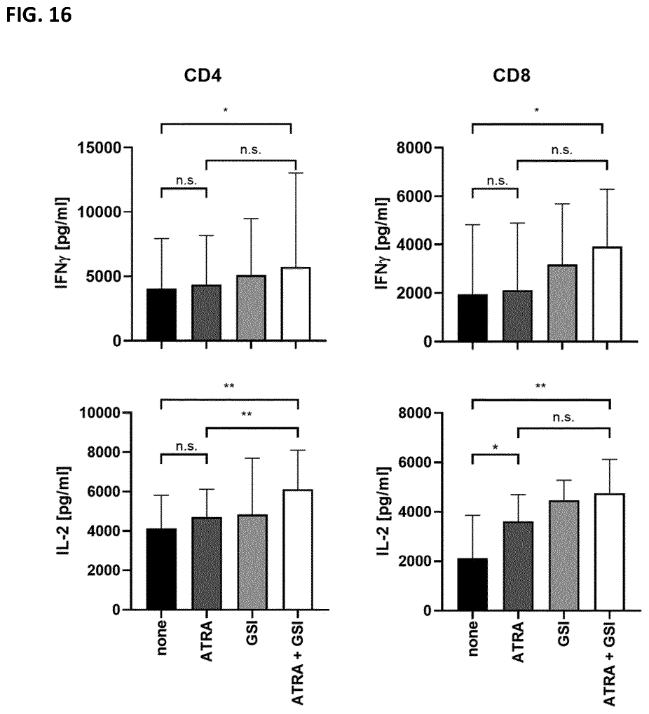

The inventors sought to determine whether the increase in BCMA expression that is induced by ATRA treatment, affected the anti-myeloma reactivity of BCMA-CAR T-cells. First, the inventors confirmed that ATRA treatment had no negative effect on the viability of BCMA-CAR T-cells (FIG. 11), and did not diminish expression of the EGFRt_BCMA-CAR transgene (FIG. 12). Then, the inventors tested the cytolytic activity of BCMA-CAR T-cells and found superior cytolysis of ATRA-treated MM.1S myeloma cells compared to non-treated MM.1S myeloma cells (FIG. 13). Furthermore, the cyotolytic effect of BCMA-CAR T-cells could be further enhanced when the MM.1S target cells were previously treated with a combination of ATRA and GSI (FIG. 13). Similar results were obtained for OPM-2 cells (FIG. 14). Additionally, BCMA-CAR T-cells showed enhanced proliferative capacity (FIG. 15) and cytokine release (FIG. 16), when the target cells were pretreated with ATRA alone or a combination of ATRA and GSI.

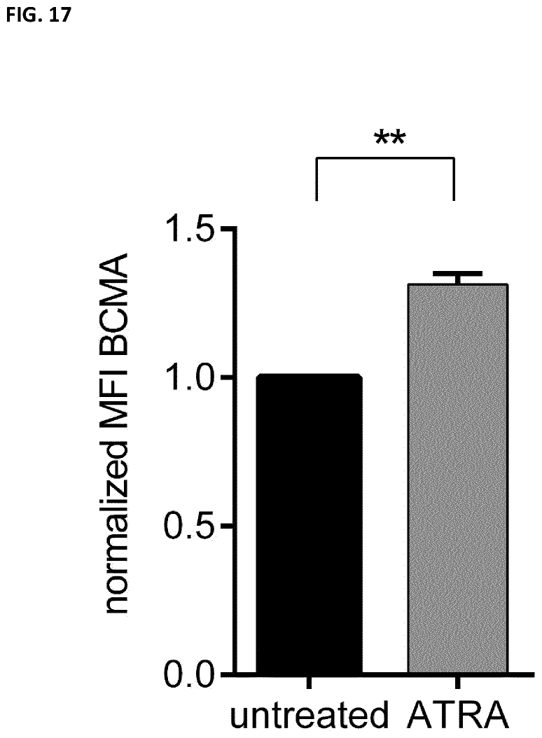

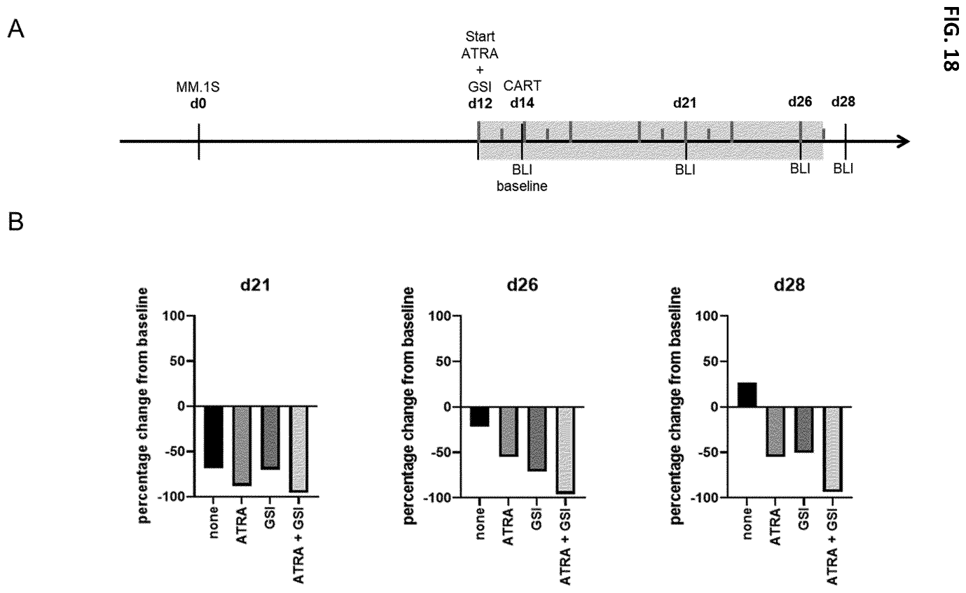

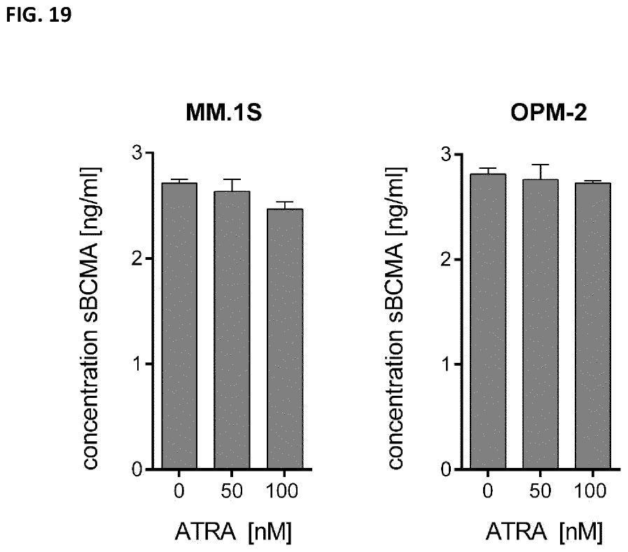

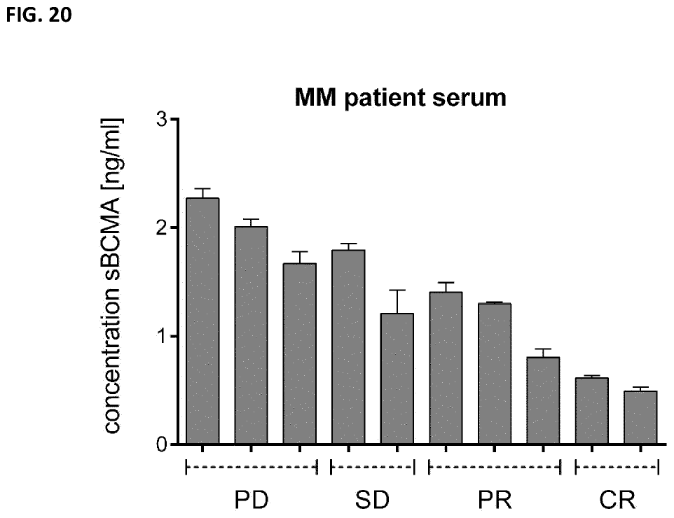

This encouraged experiments in a murine xenograft model of myeloma (NSG/MM1.S). In a first set of experiments, n=6 mice were inoculated with MM1.S cells (2×10⁶ cells given i.v. by tail vein injection) for 12 days to establish systemic myeloma, and then administered a 4-day treatment course with either ATRA (n=3 mice; 30 mg/kg given i.p. every day) or solvent control (n=3 mice). The following day, mice were sacrificed, MM.1S myeloma cells were isolated from bone marrow and BCMA expression analyzed by flow cytometry. Significantly higher BCMA expression was found on MM.1S myeloma cells from mice that had received ATRA compared to control mice (P=0.002, FIG. 17). The in vivo upregulation of BCMA after GSI treatment was shown by Pont et. al. in 2019⁴⁰.

In a second MM.1S/NSG mouse experiment, the anti-myeloma efficacy of a suboptimal dose of BCMA-CAR T-cells (1×10⁶ total CAR-T cells, CD8:CD4 at 1:1 ratio, given i.v. by tail vein injection on day 14) was investigated in combination with ATRA alone, GSI alone or a combination of both drugs. 30 mg/kg of ATRA was administrated i.p. twelve times within 16 days, starting twelve days after tumor inoculation. 1 mg/kg GSI was administrated i.p. seven times within the same time span (day 12 to day 28 after tumor inoculation).

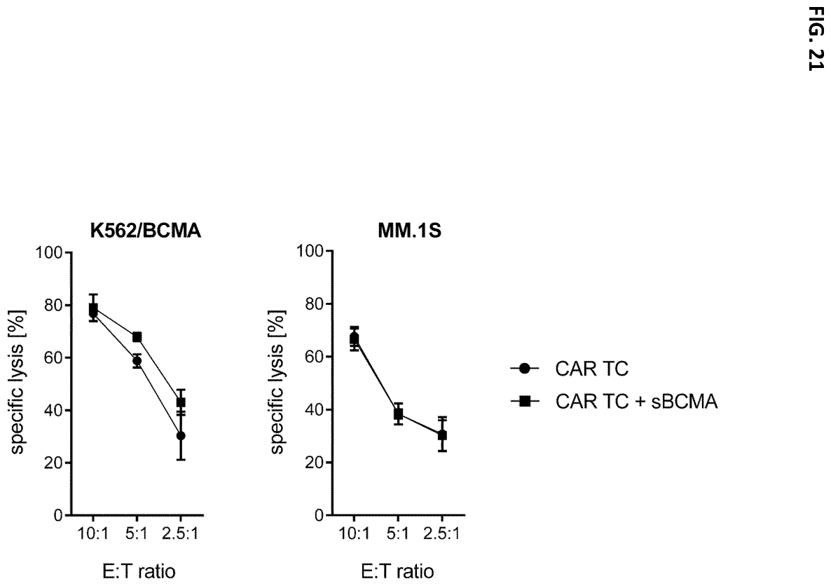

During the first days after CAR T-cell injection, bioluminescence imaging decreased in all mice groups. However, the mice which just received CAR-T cells relapsed within two weeks after the treatment. Mice treated with a combination of ATRA and BCMA-CAR T-cells relapsed much slower in comparison (FIG. 18). Furthermore, bioluminescence imaging revealed a more distinct and more stable tumor reduction, when mice were treated with a combination of CAR T-cells, ATRA and GSI. These mice achieved and remained in complete remission during the follow-up period (FIG. 18)

In aggregate, these data show that ATRA elevates BCMA expression on myeloma cells in vivo, and augments the anti-myeloma reactivity of BCMA-CAR T-cells. Additionally, BCMA-targeting immunotherapies can benefit not only from treatment with ATRA alone, but even more from a combination treatment with GSI and ATRA.

### Example 5: sBCMA Does Not Compromise BCMA-CAR T-Cell Function Against ATRA-Treated Myeloma Cells

It is well established that the extracellular portion of membrane-bound BCMA can be shed from myeloma cells to release a shorter, soluble BCMA (sBCMA) protein isoform^{26, 27}. The inventors measured sBCMA and in the supernatants of MM.1S and OPM-2 myeloma cells that had been treated with ATRA for 72 hours and obtained similar values as in the corresponding non-treated cell lines (FIG. 19). Notably, the concentration of sBCMA in conditioned medium of ATRA-treated or untreated MM.1S and OPM-2 myeloma cells was higher than in serum from myeloma patients (FIG. 20). The inventors analyzed the cytolytic activity of BCMA-CAR T-cells in fresh or sBCMA-containing medium and observed similarly potent cytolytic activity against MM.1S or K562/BCMA target cells at all effector to target cell ratios and time points (FIG. 21). These data show that ATRA treatment does not accelerate the release of sBCMA from myeloma cells and that the increased reactivity of the presently used BCMA-CAR T-cells against ATRA-treated myeloma cells is not diminished through interference from sBCMA.

Collectively, these data demonstrate that ATRA induces increased BCMA expression in primary myeloma cells and myeloma cell lines, and enables enhanced reactivity of BCMA-CAR T-cells in vitro and in vivo. These data encourage the investigation of BCMA-CAR T-cells and other BCMA-directed immunotherapies in combination with ATRA. This effect can be potentiated by combining ATRA with a GSI.

Ongoing clinical trials with BCMA-CAR T-cells have shown first promising results in MM patients, raising high expectations in this treatment strategy^{16, 19}. However, despite high initial response rates tumor eradication remained incomplete in some of the patients, the overall duration of response was short and there were case reports of relapses after BCMA downregulation or loss^{16, 17}. This phenomenon has also been described in other CAR T-cell trials targeting CD19 and CD22. There is strong evidence that diminished antigen densities might be the mechanism of tumor escape from CAR-targeted therapies³²⁻³⁵.

Furthermore, the baseline expression of BCMA can be low and non-uniform on MM cells, leading to the exclusion of patients from the treatment, or suboptimal response to the treatment^{16, 17}. In accordance with previous reports, highly variable BCMA expression levels were found in MM samples^{36, 37}. It was further observed that BCMA molecules are equally expressed on the surface of primary myeloma cells from ND and R/R MM patients, indicating that BCMA-CAR therapy is applicable for both disease conditions.

For these reasons, there is the need to enhance BCMA-CAR T-cell efficacy by increasing BCMA density on the surface of the target cells. It has been demonstrated that the retinoic acid receptor on MM cells plays an important role in the induction of CD38 expression by ATRA^{22, 38, 39}. Therefore, it was hypothesized that ATRA could also upregulate other MM antigens than CD38, in particular BCMA.

Indeed, these data show that BCMA gene and surface expression was increased on all tumor cell lines and primary malignant plasma cells after ATRA-treatment. Importantly, this was also true for primary myeloma cells with low BCMA baseline expression. To verify this effect in vivo, MM.1S tumor-bearing NSG mice were injected with ATRA. Analysis of these MM.1S revealed a significant increase of BCMA expression after the ATRA treatment.

It was shown before, that an increase of BCMA surface expression on target cells leads to enhanced recognition by BCMA-CAR T-cells⁴⁰. Enhanced anti-myeloma efficacy of BCMA-CAR T-cells after BCMA upregulation by ATRA treatment was confirmed. This synergistic effect between CAR T-cell therapy and ATRA could be used as strategy to counteract the outgrowth of antigen-low tumor cell clones, sustaining the therapeutic efficacy of BCMA-CAR T-cells. Furthermore, patients with low BCMA baseline expression could be treated with ATRA and then successfully with BCMA-CAR T-cells. Additionally, BCMA expression on tumor cells could be further enhanced by combining ATRA with GSI administration.

The inventors analyzed serum samples from MM patients for sBCMA and found a correlation between the concentration of soluble BCMA and the disease status. In line with previous reports, the serum sBCMA levels were higher among patients with progressive disease than in patients with a therapeutic response to immunomodulatory or proteasome inhibitor therapy, or low tumor burden²⁷.

The inventors examined if treatment with ATRA also leads to increased sBCMA levels in the supernatant of myeloma cell lines. Despite significantly enhanced levels of membrane bound BCMA, they observed no rise of sBCMA in the supernatant of drug exposed cells. This leads to the conclusion that there is no immediate increase in ectodomain shedding after expressing more membrane-bound molecules.

Nevertheless, the inventors wanted to know whether sBCMA could abrogate the anti-myeloma function of these BCMA-CAR T-cells in principle. Therefore, they tested CAR T-cell functionality in the presence of up to 150 ng/ml sBCMA, which is about ten times the average concentration the inventors observed in the serum of patients with progressive disease. Even with this high concentration the inventors could not find sBCMA having a negative impact on the cytolytic activity of these BCMA-CAR T-cells.

There are divergent prior reports on the impact of sBCMA on BCMA-CAR T-cells. M. J. Pont et al. reported that CAR T-cell cytokine release and proliferation are impaired by even low levels of 10 ng/ml sBCMA and cytotoxicity at high sBCMA levels of at least 100 ng/ml⁴⁰. On the other hand, the groups of R. O. Carpenter et al and K. M. Friedman et al. found that BCMA-CAR T-cells were highly efficient in MM xenograft mice, despite increased sBCMA serum levels of about 7 ng/ml^{37, 41}. Furthermore, R. O. Carpenter et al. found that sBCMA with concentrations up to 150 ng/ml had no impact on CAR T-cell cytokine release in vitro⁴¹. These different observations might be due to the use of different BCMA CARs, binding to distinct epitopes. Not all of these epitopes might be accessible in the soluble BCMA conformation.

In conclusion, this study demonstrates that the efficacy of BCMA-CAR T-cell therapy can be improved by up-regulation of antigen expression with ATRA. Therefore, according to the invention, retinoids such as ATRA can be used synergistically with BCMA-CAR T-cells in a clinical setting to increase response rates and extend duration of responses in ND and R/R myeloma. The use of a well-chosen CAR construct might reduce negative impacts by sBCMA molecules in the serum of patients. The effect of BCMA up-regulation and BCMA-CAR T-cell targeting is even greater when not only using ATRA, but a combination of ATRA and GSI.

### INDUSTRIAL APPLICABILITY

The immunotherapeutic agent and retinoids as used according to the invention, can be industrially manufactured and sold as products for the claimed methods and uses (e.g. for treating a cancer as defined herein), in accordance with known standards for the manufacture of pharmaceutical and diagnostic products. Accordingly, the present invention is industrially applicable.

### SEQUENCES

All nucleotide sequences are indicated in a 5′-to-3′ order. All amino acid sequences are indicated in an N-to-C-terminal order using the three-letter amino acid code.

Whole nucleotide sequence of the chimeric antigen receptor (CAR) capable of binding
to BCMA used in the Examples (SEQ ID NO: 1):
ATGCTGCTGCTCGTGACATCTCTGCTGCTGTGCGAGCTGCCCCACCCCGCCTTTCTGCT
GATTCCT
CAGGTGCAGCTGGTGCAGTCTGGCGCCGAAGTGAAGAAACCAGGCGCCAGCGTGAAG
GTGTCCTGCAAGGCCAGCGGCTACAGCTTCCCCGACTACTACATCAACTGGGTGCGCC
AGGCCCCTGGACAGGGCCTGGAATGGATGGGCTGGATCTACTTCGCCAGCGGCAACT
CCGAGTACAACCAGAAATTCACCGGCAGAGTGACCATGACCCGGGACACCAGCATCAA
CACCGCCTACATGGAACTGAGCAGCCTGACCAGCGAGGATACCGCCGTGTACTTCTGC
GCCAGCCTGTACGACTACGACTGGTACTTCGACGTGTGGGGCCAGGGCACAATGGTCA
CCGTGTCTAGC
GGAGGCGGAGGCTCCGGAGGGGGAGGATCTGGGGGAGGCGGAAGC
GATATCGTGATGACCCAGACCCCCCTGAGCCTGAGCGTGACACCTGGACAGCCTGCCA
GCATCAGCTGCAAGAGCAGCCAGAGCCTGGTGCACAGCAACGGCAACACCTACCTGCA
CTGGTATCTGCAGAAGCCCGGCCAGAGCCCCCAGCTGCTGATCTACAAGGTGTCCAAC
CGGTTCAGCGGCGTGCCCGACAGATTTTCTGGCAGCGGCTCCGGCACCGACTTCACCC
TGAAGATCTCCCGGGTGGAAGCCGAGGACGTGGGCATCTACTACTGCAGCCAGTCCAG
CATCTACCCCTGGACCTTCGGCCAGGGGACCAAGCTGGAAATCAAA
AAAGAGTCTAAGTACGGACCGCCTTGTCCTCCTTGTCCAGCTCCTCCTGTGGCCGGAC
CTAGCGTGTTCCTGTTCCCCCCAAAGCCCAAGGACACCCTGATGATCAGCCGGACCCC
CGAAGTGACCTGCGTGGTGGTGGATGTGTCCCAGGAAGATCCCGAGGTGCAGTTCAAT
TGGTACGTGGACGGCGTGGAAGTGCACAACGCCAAGACCAAGCCCAGAGAGGAACAG
TTCCAGAGCACCTACCGGGTGGTGTCCGTGCTGACAGTGCTGCACCAGGACTGGCTGA
ACGGCAAAGAGTACAAGTGCAAGGTGTCCAACAAGGGCCTGCCCAGCAGCATCGAGAA
AACCATCAGCAAGGCCAAGGGCCAGCCTCGCGAGCCCCAGGTGTACACACTGCCTCCA
AGCCAGGAAGAGATGACCAAGAACCAGGTGTCCCTGACCTGTCTCGTGAAGGGCTTCT
ACCCCAGCGACATTGCCGTGGAATGGGAGAGCAACGGCCAGCCCGAGAACAACTACAA
GACCACCCCCCCTGTGCTGGACAGCGACGGCTCATTCTTCCTGTACAGCAGACTGACC
GTGGACAAGAGCCGGTGGCAGGAAGGCAACGTGTTCAGCTGCAGCGTGATGCACGAG
GCCCTGCACAACCACTACACCCAGAAGTCCCTGTCTCTGAGCCTGGGCAAG
ATGTTCTGGGTGCTGGTGGTCGTGGGCGGAGTGCTGGCCTGTTACAGCCTGCTCGTGA
CCGTGGCCTTCATCATCTTTTGGGTC
AAGCGGGGCAGAAAGAAGCTGCTGTATATCTTCAAGCAGCCCTTCATGCGGCCCGTGC
AGACCACACAGGAAGAGGACGGCTGCTCCTGCCGGTTCCCCGAGGAAGAAGAAGGCG
GCTGCGAGCTG
AGAGTGAAGTTCAGCAGAAGCGCCGACGCCCCTGCCTATCAGCAGGGCCAGAACCAG
CTGTACAACGAGCTGAACCTGGGCAGACGGGAAGAGTACGACGTGCTGGATAAGCGGA
GAGGCCGGGACCCTGAGATGGGCGGCAAGCCTAGAAGAAAGAACCCCCAGGAAGGCC
TGTATAACGAACTGCAGAAAGACAAGATGGCCGAGGCCTACAGCGAGATCGGAATGAA
GGGCGAGCGGAGAAGAGGCAAGGGCCACGATGGCCTGTACCAGGGACTGAGCACCG
CCACCAAGGATACCTATGACGCACTGCACATGCAGGCCCTGCCCCCCAGA
CTCGAGGGGGGAGGCGAAGGCAGAGGATCTCTGCTGACATGCGGCGACGTGGAAGAG
AACCCTGGCCCCAGA
ATGCTGCTGCTCGTGACAAGCCTGCTGCTGTGCGAGCTGCCCCACCCTGCCTTTCTGC
TGATCCCC
CGGAAAGTGTGCAACGGCATCGGCATCGGAGAGTTCAAGGACAGCCTGTCCATCAACG
CCACCAACATCAAGCACTTCAAGAATTGCACCAGCATCAGCGGCGACCTGCACATCCTG
CCAGTGGCCTTTAGAGGCGACAGCTTCACCCACACCCCCCCACTGGATCCACAGGAAC
TGGATATTCTGAAAACCGTAAAGGAAATCACAGGGTTTTTGCTGATTCAGGCTTGGCCT
GAAAACAGGACGGACCTCCATGCCTTTGAGAACCTAGAAATCATACGCGGCAGGACCA
AGCAACATGGTCAGTTTTCTCTTGCAGTCGTCAGCCTGAACATAACATCCTTGGGATTAC
GCTCCCTCAAGGAGATAAGTGATGGAGATGTGATAATTTCAGGAAACAAAAATTTGTGCT
ATGCAAATACAATAAACTGGAAAAAACTGTTTGGGACCTCCGGTCAGAAAACCAAAATTA
TAAGCAACAGAGGTGAAAACAGCTGCAAGGCCACAGGCCAGGTCTGCCATGCCTTGTG
CTCCCCCGAGGGCTGCTGGGGCCCGGAGCCCAGGGACTGCGTCTCTTGCCGGAATGT
CAGCCGAGGCAGGGAATGCGTGGACAAGTGCAACCTTCTGGAGGGTGAGCCAAGGGA
GTTTGTGGAGAACTCTGAGTGCATACAGTGCCACCCAGAGTGCCTGCCTCAGGCCATG
AACATCACCTGCACAGGACGGGGACCAGACAACTGTATCCAGTGTGCCCACTACATTGA
CGGCCCCCACTGCGTCAAGACCTGCCCGGCAGGAGTCATGGGAGAAAACAACACCCT
GGTCTGGAAGTACGCAGACGCCGGCCATGTGTGCCACCTGTGCCATCCAAACTGCACC
TACGGATGCACTGGGCCAGGTCTTGAAGGCTGTCCAACGAATGGGCCTAAGATCCCGT
CCATCGCCACTGGGATGGTGGGGGCCCTCCTCTTGCTGCTGGTGGTGGCCCTGGGGA
TCGGCCTCTTCATGTGA
Nucleotide sequence of the GMCSF signal peptide (SEQ ID NO: 2):
ATGCTGCTGCTCGTGACATCTCTGCTGCTGTGCGAGCTGCCCCACCCCGCCTTTCTGCTGATTCCT
Nucleotide sequence of the BCMA single chain variable fragment VH (SEQ ID NO: 3):
CAGGTGCAGCTGGTGCAGTCTGGCGCCGAAGTGAAGAAACCAGGCGCCAGCGTGAAGGTGTCCT
GCAAGGCCAGCGGCTACAGCTTCCCCGACTACTACATCAACTGGGTGCGCCAGGCCCCTGGACA
GGGCCTGGAATGGATGGGCTGGATCTACTTCGCCAGCGGCAACTCCGAGTACAACCAGAAATTCA
CCGGCAGAGTGACCATGACCCGGGACACCAGCATCAACACCGCCTACATGGAACTGAGCAGCCT
GACCAGCGAGGATACCGCCGTGTACTTCTGCGCCAGCCTGTACGACTACGACTGGTACTTCGACG
TGTGGGGCCAGGGCACAATGGTCACCGTGTCTAGC
Nucleotide sequence of the (4GS)3 linker (SEQ ID NO: 4):
GGAGGCGGAGGCTCCGGAGGGGGAGGATCTGGGGGAGGCGGAAGC
Nucleotide sequence of the BCMA single chain variable fragment VL (SEQ ID NO: 5):
GATATCGTGATGACCCAGACCCCCCTGAGCCTGAGCGTGACACCTGGACAGCCTGCCAGCATCAG
CTGCAAGAGCAGCCAGAGCCTGGTGCACAGCAACGGCAACACCTACCTGCACTGGTATCTGCAGA
AGCCCGGCCAGAGCCCCCAGCTGCTGATCTACAAGGTGTCCAACCGGTTCAGCGGCGTGCCCGA
CAGATTTTCTGGCAGCGGCTCCGGCACCGACTTCACCCTGAAGATCTCCCGGGTGGAAGCCGAGG
ACGTGGGCATCTACTACTGCAGCCAGTCCAGCATCTACCCCTGGACCTTCGGCCAGGGGACCAAG
CTGGAAATCAAA
Nucleotide sequence of the IgG4-Fc Hinge-CH2-CH3 4/2NQ (SEQ ID NO: 6):
AAAGAGTCTAAGTACGGACCGCCTTGTCCTCCTTGTCCAGCTCCTCCTGTGGCCGGACCTAGCGT
GTTCCTGTTCCCCCCAAAGCCCAAGGACACCCTGATGATCAGCCGGACCCCCGAAGTGACCTGCG
TGGTGGTGGATGTGTCCCAGGAAGATCCCGAGGTGCAGTTCAATTGGTACGTGGACGGCGTGGAA
GTGCACAACGCCAAGACCAAGCCCAGAGAGGAACAGTTCCAGAGCACCTACCGGGTGGTGTCCG
TGCTGACAGTGCTGCACCAGGACTGGCTGAACGGCAAAGAGTACAAGTGCAAGGTGTCCAACAAG
GGCCTGCCCAGCAGCATCGAGAAAACCATCAGCAAGGCCAAGGGCCAGCCTCGCGAGCCCCAGG
TGTACACACTGCCTCCAAGCCAGGAAGAGATGACCAAGAACCAGGTGTCCCTGACCTGTCTCGTG
AAGGGCTTCTACCCCAGCGACATTGCCGTGGAATGGGAGAGCAACGGCCAGCCCGAGAACAACTA
CAAGACCACCCCCCCTGTGCTGGACAGCGACGGCTCATTCTTCCTGTACAGCAGACTGACCGTGG
ACAAGAGCCGGTGGCAGGAAGGCAACGTGTTCAGCTGCAGCGTGATGCACGAGGCCCTGCACAA
CCACTACACCCAGAAGTCCCTGTCTCTGAGCCTGGGCAAG
Nucleotide sequence of the CD28 transmembrane domain (SEQ ID NO: 7):
ATGTTCTGGGTGCTGGTGGTCGTGGGCGGAGTGCTGGCCTGTTACAGCCTGCTCGTGACCGTGG
CCTTCATCATCTTTTGGGTC
Nucleotide sequence of the 4-1BB cytoplasmic domain (SEQ ID NO: 8):
AAGCGGGGCAGAAAGAAGCTGCTGTATATCTTCAAGCAGCCCTTCATGCGGCCCGTGCAGACCAC
ACAGGAAGAGGACGGCTGCTCCTGCCGGTTCCCCGAGGAAGAAGAAGGCGGCTGCGAGCTG
Nucleotide sequence of the CD3 zeta domain (SEQ ID NO: 9):
AGAGTGAAGTTCAGCAGAAGCGCCGACGCCCCTGCCTATCAGCAGGGCCAGAACCAGCTGTACAA
CGAGCTGAACCTGGGCAGACGGGAAGAGTACGACGTGCTGGATAAGCGGAGAGGCCGGGACCCT
GAGATGGGCGGCAAGCCTAGAAGAAAGAACCCCCAGGAAGGCCTGTATAACGAACTGCAGAAAGA
CAAGATGGCCGAGGCCTACAGCGAGATCGGAATGAAGGGCGAGCGGAGAAGAGGCAAGGGCCA
CGATGGCCTGTACCAGGGACTGAGCACCGCCACCAAGGATACCTATGACGCACTGCACATGCAGG
CCCTGCCCCCCAGA
Nucleotide sequence of the T2A ribosomal skip element (SEQ ID NO: 10):
CTCGAGGGCGGAGGCGAAGGCAGAGGATCTCTGCTGACATGCGGCGACGTGGAAGAGAACCCTG
GCCCCAGA
Nucleotide sequence of the GMCSF signal peptide (SEQ ID NO: 11):
ATGCTGCTGCTCGTGACAAGCCTGCTGCTGTGCGAGCTGCCCCACCCTGCCTTTCTGCTGATCCC
C
Nucleotide sequence of the tEGFR sequence (SEQ ID NO: 12):
CGGAAAGTGTGCAACGGCATCGGCATCGGAGAGTTCAAGGACAGCCTGTCCATCAACGCCACCAA
CATCAAGCACTTCAAGAATTGCACCAGCATCAGCGGCGACCTGCACATCCTGCCAGTGGCCTTTAG
AGGCGACAGCTTCACCCACACCCCCCCACTGGATCCACAGGAACTGGATATTCTGAAAACCGTAAA
GGAAATCACAGGGTTTTTGCTGATTCAGGCTTGGCCTGAAAACAGGACGGACCTCCATGCCTTTGA
GAACCTAGAAATCATACGCGGCAGGACCAAGCAACATGGTCAGTTTTCTCTTGCAGTCGTCAGCCT
GAACATAACATCCTTGGGATTACGCTCCCTCAAGGAGATAAGTGATGGAGATGTGATAATTTCAGG
AAACAAAAATTTGTGCTATGCAAATACAATAAACTGGAAAAAACTGTTTGGGACCTCCGGTCAGAAA
ACCAAAATTATAAGCAACAGAGGTGAAAACAGCTGCAAGGCCACAGGCCAGGTCTGCCATGCCTT
GTGCTCCCCCGAGGGCTGCTGGGGCCCGGAGCCCAGGGACTGCGTCTCTTGCCGGAATGTCAGC
CGAGGCAGGGAATGCGTGGACAAGTGCAACCTTCTGGAGGGTGAGCCAAGGGAGTTTGTGGAGA
ACTCTGAGTGCATACAGTGCCACCCAGAGTGCCTGCCTCAGGCCATGAACATCACCTGCACAGGA
CGGGGACCAGACAACTGTATCCAGTGTGCCCACTACATTGACGGCCCCCACTGCGTCAAGACCTG
CCCGGCAGGAGTCATGGGAGAAAACAACACCCTGGTCTGGAAGTACGCAGACGCCGGCCATGTG
TGCCACCTGTGCCATCCAAACTGCACCTACGGATGCACTGGGCCAGGTCTTGAAGGCTGTCCAAC
GAATGGGCCTAAGATCCCGTCCATCGCCACTGGGATGGTGGGGGCCCTCCTCTTGCTGCTGGTG
GTGGCCCTGGGGATCGGCCTCTTCATGTGA
Whole amino acid sequence of the chimeric antigen receptor (CAR) capable of binding to BCMA
used in the Examples (SEQ ID NO: 13):
Met Leu Leu Leu Val Thr Ser Leu Leu Leu Cys Glu Leu Pro His Pro Ala Phe Leu Leu Ile Pro
Gln Val Gln Leu Val Gln Ser Gly Ala Glu Val Lys Lys Pro Gly Ala Ser Val Lys Val Ser Cys Lys Ala Ser
Gly Tyr Ser Phe Pro Asp Tyr Tyr Ile Asn Trp Val Arg Gln Ala Pro Gly Gln Gly Leu Glu Trp Met Gly Trp
Ile Tyr Phe Ala Ser Gly Asn Ser Glu Tyr Asn Gln Lys Phe Thr Gly Arg Val Thr Met Thr Arg Asp Thr Ser
Ile Asn Thr Ala Tyr Met Glu Leu Ser Ser Leu Thr Ser Glu Asp Thr Ala Val Tyr Phe Cys Ala Ser Leu Tyr
Asp Tyr Asp Trp Tyr Phe Asp Val Trp Gly Gln Gly Thr Met Val Thr Val Ser Ser
Gly Gly Gly Gly Ser Gly Gly Gly Gly Ser Gly Gly Gly Gly Ser
Asp Ile Val Met Thr Gln Thr Pro Leu Ser Leu Ser Val Thr Pro Gly Gln Pro Ala Ser Ile Ser Cys Lys Ser
Ser GIn Ser Leu Val His Ser Asn Gly Asn Thr Tyr Leu His Trp Tyr Leu Gln Lys Pro Gly Gln Ser Pro Gln
Leu Leu Ile Tyr Lys Val Ser Asn Arg Phe Ser Gly Val Pro Asp Arg Phe Ser Gly Ser Gly Ser Gly Thr Asp
Phe Thr Leu Lys Ile Ser Arg Val Glu Ala Glu Asp Val Gly Ile Tyr Tyr Cys Ser Gln Ser Ser Ile Tyr Pro
Trp Thr Phe Gly Gln Gly Thr Lys Leu Glu Ile Lys
Lys Glu Ser Lys Tyr Gly Pro Pro Cys Pro Pro Cys Pro Ala Pro Pro Val Ala Gly Pro Ser Val Phe Leu Phe
Pro Pro Lys Pro Lys Asp Thr Leu Met Ile Ser Arg Thr Pro Glu Val Thr Cys Val Val Val Asp Val Ser Gln
Glu Asp Pro Glu Val Gln Phe Asn Trp Tyr Val Asp Gly Val Glu Val His Asn Ala Lys Thr Lys Pro Arg Glu
Glu GIn Phe Gln Ser Thr Tyr Arg Val Val Ser Val Leu Thr Val Leu His Gln Asp Trp Leu Asn Gly Lys Glu
Tyr Lys Cys Lys Val Ser Asn Lys Gly Leu Pro Ser Ser Ile Glu Lys Thr Ile Ser Lys Ala Lys Gly Gln Pro
Arg Glu Pro GIn Val Tyr Thr Leu Pro Pro Ser Gln Glu Glu Met Thr Lys Asn Gln Val Ser Leu Thr Cys Leu
Val Lys Gly Phe Tyr Pro Ser Asp Ile Ala Val Glu Trp Glu Ser Asn Gly Gln Pro Glu Asn Asn Tyr Lys Thr
Thr Pro Pro Val Leu Asp Ser Asp Gly Ser Phe Phe Leu Tyr Ser Arg Leu Thr Val Asp Lys Ser Arg Trp
Gln Glu Gly Asn Val Phe Ser Cys Ser Val Met His Glu Ala Leu His Asn His Tyr Thr Gln Lys Ser Leu Ser
Leu Ser Leu Gly Lys
Met Phe Trp Val Leu Val Val Val Gly Gly Val Leu Ala Cys Tyr Ser Leu Leu Val Thr Val Ala Phe Ile Ile
Phe Trp Val
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro GIn Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr
Tyr Asp Ala Leu His Met Gln Ala Leu Pro Pro Arg
Leu Glu Gly Gly Gly Glu Gly Arg Gly Ser Leu Leu Thr Cys Gly Asp Val Glu Glu Asn Pro Gly Pro Arg
Met Leu Leu Leu Val Thr Ser Leu Leu Leu Cys Glu Leu Pro His Pro Ala Phe Leu Leu Ile Pro
Arg Lys Val Cys Asn Gly Ile Gly Ile Gly Glu Phe Lys Asp Ser Leu Ser Ile Asn Ala Thr Asn Ile Lys His
Phe Lys Asn Cys Thr Ser Ile Ser Gly Asp Leu His Ile Leu Pro Val Ala Phe Arg Gly Asp Ser Phe Thr His
Thr Pro Pro Leu Asp Pro GIn Glu Leu Asp Ile Leu Lys Thr Val Lys Glu Ile Thr Gly Phe Leu Leu Ile Gln
Ala Trp Pro Glu Asn Arg Thr Asp Leu His Ala Phe Glu Asn Leu Glu Ile Ile Arg Gly Arg Thr Lys GIn His
Gly Gln Phe Ser Leu Ala Val Val Ser Leu Asn Ile Thr Ser Leu Gly Leu Arg Ser Leu Lys Glu Ile Ser Asp
Gly Asp Val Ile Ile Ser Gly Asn Lys Asn Leu Cys Tyr Ala Asn Thr Ile Asn Trp Lys Lys Leu Phe Gly Thr
Ser Gly Gln Lys Thr Lys Ile Ile Ser Asn Arg Gly Glu Asn Ser Cys Lys Ala Thr Gly Gln Val Cys His Ala
Leu Cys Ser Pro Glu Gly Cys Trp Gly Pro Glu Pro Arg Asp Cys Val Ser Cys Arg Asn Val Ser Arg Gly
Arg Glu Cys Val Asp Lys Cys Asn Leu Leu Glu Gly Glu Pro Arg Glu Phe Val Glu Asn Ser Glu Cys Ile
Gln Cys His Pro Glu Cys Leu Pro GIn Ala Met Asn Ile Thr Cys Thr Gly Arg Gly Pro Asp Asn Cys Ile Gln
Cys Ala His Tyr Ile Asp Gly Pro His Cys Val Lys Thr Cys Pro Ala Gly Val Met Gly Glu Asn Asn Thr Leu
Val Trp Lys Tyr Ala Asp Ala Gly His Val Cys His Leu Cys His Pro Asn Cys Thr Tyr Gly Cys Thr Gly Pro
Gly Leu Glu Gly Cys Pro Thr Asn Gly Pro Lys Ile Pro Ser Ile Ala Thr Gly Met Val Gly Ala Leu Leu Leu
Leu Leu Val Val Ala Leu Gly Ile Gly Leu Phe Met
Amino acid sequence of the GMCSF signal peptide (SEQ ID NO: 14):
Met Leu Leu Leu Val Thr Ser Leu Leu Leu Cys Glu Leu Pro His Pro Ala Phe Leu Leu Ile Pro
Amino acid sequence of the BCMA single chain variable fragment VH (SEQ ID NO: 15):
Gln Val Gln Leu Val Gln Ser Gly Ala Glu Val Lys Lys Pro Gly Ala Ser Val Lys Val Ser Cys Lys Ala Ser
Gly Tyr Ser Phe Pro Asp Tyr Tyr Ile Asn Trp Val Arg Gln Ala Pro Gly Gln Gly Leu Glu Trp Met Gly Trp
Ile Tyr Phe Ala Ser Gly Asn Ser Glu Tyr Asn Gln Lys Phe Thr Gly Arg Val Thr Met Thr Arg Asp Thr Ser
Ile Asn Thr Ala Tyr Met Glu Leu Ser Ser Leu Thr Ser Glu Asp Thr Ala Val Tyr Phe Cys Ala Ser Leu Tyr
Asp Tyr Asp Trp Tyr Phe Asp Val Trp Gly Gln Gly Thr Met Val Thr Val Ser Ser
Amino acid sequence of the (4GS)3 linker (SEQ ID NO: 16):
Gly Gly Gly Gly Ser Gly Gly Gly Gly Ser Gly Gly Gly Gly Ser
Amino acid sequence of the BCMA single chain variable fragment VL (SEQ ID NO: 17):
Asp Ile Val Met Thr Gln Thr Pro Leu Ser Leu Ser Val Thr Pro Gly Gln Pro Ala Ser Ile Ser Cys Lys Ser
Ser Gln Ser Leu Val His Ser Asn Gly Asn Thr Tyr Leu His Trp Tyr Leu Gln Lys Pro Gly Gln Ser Pro Gln
Leu Leu Ile Tyr Lys Val Ser Asn Arg Phe Ser Gly Val Pro Asp Arg Phe Ser Gly Ser Gly Ser Gly Thr Asp
Phe Thr Leu Lys Ile Ser Arg Val Glu Ala Glu Asp Val Gly Ile Tyr Tyr Cys Ser Gln Ser Ser Ile Tyr Pro
Trp Thr Phe Gly Gln Gly Thr Lys Leu Glu Ile Lys
Amino acid sequence of the lgG4-Fc Hinge-CH2-CH3 4/2NQ (SEQ ID NO: 18):
Lys Glu Ser Lys Tyr Gly Pro Pro Cys Pro Pro Cys Pro Ala Pro Pro Val Ala Gly Pro Ser Val Phe Leu Phe
Pro Pro Lys Pro Lys Asp Thr Leu Met Ile Ser Arg Thr Pro Glu Val Thr Cys Val Val Val Asp Val Ser Gln
Glu Asp Pro Glu Val Gln Phe Asn Trp Tyr Val Asp Gly Val Glu Val His Asn Ala Lys Thr Lys Pro Arg Glu
Glu GIn Phe Gln Ser Thr Tyr Arg Val Val Ser Val Leu Thr Val Leu His Gln Asp Trp Leu Asn Gly Lys Glu
Tyr Lys Cys Lys Val Ser Asn Lys Gly Leu Pro Ser Ser Ile Glu Lys Thr Ile Ser Lys Ala Lys Gly Gln Pro
Arg Glu Pro GIn Val Tyr Thr Leu Pro Pro Ser Gln Glu Glu Met Thr Lys Asn Gln Val Ser Leu Thr Cys Leu
Val Lys Gly Phe Tyr Pro Ser Asp Ile Ala Val Glu Trp Glu Ser Asn Gly Gln Pro Glu Asn Asn Tyr Lys Thr
Thr Pro Pro Val Leu Asp Ser Asp Gly Ser Phe Phe Leu Tyr Ser Arg Leu Thr Val Asp Lys Ser Arg Trp
Gln Glu Gly Asn Val Phe Ser Cys Ser Val Met His Glu Ala Leu His Asn His Tyr Thr Gln Lys Ser Leu Ser
Leu Ser Leu Gly Lys
Amino acid sequence of the CD28 transmembrane domain (SEQ ID NO: 19):
Met Phe Trp Val Leu Val Val Val Gly Gly Val Leu Ala Cys Tyr Ser Leu Leu Val Thr Val Ala Phe Ile Ile
Phe Trp Val
Amino acid sequence of the 4-1BB cytoplasmic domain (SEQ ID NO: 20):
Lys Arg Gly Arg Lys Lys Leu Leu Tyr Ile Phe Lys Gln Pro Phe Met Arg Pro Val Gln Thr Thr Gln Glu Glu
Asp Gly Cys Ser Cys Arg Phe Pro Glu Glu Glu Glu Gly Gly Cys Glu Leu
Amino acid sequence of the CD3 zeta domain (SEQ ID NO: 21):
Arg Val Lys Phe Ser Arg Ser Ala Asp Ala Pro Ala Tyr Gln Gln Gly Gln Asn Gln Leu Tyr Asn Glu Leu Asn
Leu Gly Arg Arg Glu Glu Tyr Asp Val Leu Asp Lys Arg Arg Gly Arg Asp Pro Glu Met Gly Gly Lys Pro Arg
Arg Lys Asn Pro GIn Glu Gly Leu Tyr Asn Glu Leu Gln Lys Asp Lys Met Ala Glu Ala Tyr Ser Glu Ile Gly
Met Lys Gly Glu Arg Arg Arg Gly Lys Gly His Asp Gly Leu Tyr Gln Gly Leu Ser Thr Ala Thr Lys Asp Thr
Tyr Asp Ala Leu His Met Gln Ala Leu Pro Pro Arg
Amino acid sequence of the T2A ribosomal skip element (SEQ ID NO: 22):
Leu Glu Gly Gly Gly Glu Gly Arg Gly Ser Leu Leu Thr Cys Gly Asp Val Glu Glu Asn Pro Gly Pro Arg
Amino acid sequence of the GMCSF signal peptide (SEQ ID NO: 23):
Met Leu Leu Leu Val Thr Ser Leu Leu Leu Cys Glu Leu Pro His Pro Ala Phe Leu Leu Ile Pro
Amino acid sequence of the tEGFR sequence (SEQ ID NO: 24):
Arg Lys Val Cys Asn Gly Ile Gly Ile Gly Glu Phe Lys Asp Ser Leu Ser Ile Asn Ala Thr Asn Ile Lys His
Phe Lys Asn Cys Thr Ser Ile Ser Gly Asp Leu His Ile Leu Pro Val Ala Phe Arg Gly Asp Ser Phe Thr His
Thr Pro Pro Leu Asp Pro GIn Glu Leu Asp Ile Leu Lys Thr Val Lys Glu Ile Thr Gly Phe Leu Leu Ile Gln
Ala Trp Pro Glu Asn Arg Thr Asp Leu His Ala Phe Glu Asn Leu Glu Ile Ile Arg Gly Arg Thr Lys Gln His
Gly Gln Phe Ser Leu Ala Val Val Ser Leu Asn Ile Thr Ser Leu Gly Leu Arg Ser Leu Lys Glu Ile Ser Asp
Gly Asp Val Ile Ile Ser Gly Asn Lys Asn Leu Cys Tyr Ala Asn Thr Ile Asn Trp Lys Lys Leu Phe Gly Thr
Ser Gly Gln Lys Thr Lys Ile Ile Ser Asn Arg Gly Glu Asn Ser Cys Lys Ala Thr Gly Gln Val Cys His Ala
Leu Cys Ser Pro Glu Gly Cys Trp Gly Pro Glu Pro Arg Asp Cys Val Ser Cys Arg Asn Val Ser Arg Gly
Arg Glu Cys Val Asp Lys Cys Asn Leu Leu Glu Gly Glu Pro Arg Glu Phe Val Glu Asn Ser Glu Cys Ile
Gln Cys His Pro Glu Cys Leu Pro GIn Ala Met Asn Ile Thr Cys Thr Gly Arg Gly Pro Asp Asn Cys Ile Gln
Cys Ala His Tyr Ile Asp Gly Pro His Cys Val Lys Thr Cys Pro Ala Gly Val Met Gly Glu Asn Asn Thr Leu
Val Trp Lys Tyr Ala Asp Ala Gly His Val Cys His Leu Cys His Pro Asn Cys Thr Tyr Gly Cys Thr Gly Pro
Gly Leu Glu Gly Cys Pro Thr Asn Gly Pro Lys Ile Pro Ser Ile Ala Thr Gly Met Val Gly Ala Leu Leu Leu
Leu Leu Val Val Ala Leu Gly Ile Gly Leu Phe Met

In a preferred embodiment in accordance with the invention, the chimeric antigen receptor (CAR) capable of binding to BCMA is the chimeric antigen receptor (CAR) encoded by the nucleotide sequence of SEQ ID NO: 1 or by a nucleotide sequence at least 95% identical thereto. In another preferred embodiment in accordance with the invention, the chimeric antigen receptor (CAR) capable of binding to BCMA has the amino acid sequence of SEQ ID NO: 13 or an amino acid sequence at least 95% identical thereto.

### REFERENCES

1. Kohler M, Greil C, Hudecek M, Lonial S, Raje N, Wasch R et al. Current developments in immunotherapy in the treatment of multiple myeloma. Cancer 2018; 124(10): 2075-2085. doi: 10.1002/cncr.31243
2. Hudecek M, Einsele H. Myeloma CARs are rolling into the clinical arena. Blood 2016; 128(13): 1667-1668. doi: 10.1182/blood-2016-08-729467
3. Chim C S, Kumar S K, Orlowski R Z, Cook G, Richardson P G, Gertz M A et al. Management of relapsed and refractory multiple myeloma: novel agents, antibodies, immunotherapies and beyond. Leukemia. 2018 February; 32(2):252-262. doi: 10.1038/leu.2017.329
4. Lonial S, Boise L H, Kaufman J. How I treat high-risk myeloma. Blood. 2015; 126(13):1536-1543.
5. Kumar S K, Lee J H, Lahuerta J J, Morgan G, Richardson P G, Crowley J, et al. Risk of progression and survival in multiple myeloma relapsing after therapy with IMiDs and bortezomib: a multicenter international myeloma working group study. Leukemia. 2012; 26(1):149-157.
6. Davila M L, Riviere I, Wang X, Bartido S, Park J, Curran K et al. Efficacy and toxicity management of 19-28z CAR T cell therapy in B cell acute lymphoblastic leukemia. Science translational medicine 2014; 6(224): 224ra225. doi: 10.1126/scitranslmed.3008226
7. Kochenderfer J N, Dudley M E, Kassim S H, Somerville R P, Carpenter R O, Stetler-Stevenson M et al. Chemotherapy-refractory diffuse large B-cell lymphoma and indolent B-cell malignancies can be effectively treated with autologous T cells expressing an anti-CD19 chimeric antigen receptor. Journal of clinical oncology: official journal of the American Society of Clinical Oncology 2015; 33(6): 540-549. doi: 10.1200/JCO.2014.56.2025
8. Maude S L, Frey N, Shaw P A, Aplenc R, Barrett D M, Bunin N J et al. Chimeric antigen receptor T cells for sustained remissions in leukemia. The New England journal of medicine 2014; 371(16): 1507-1517. doi: 10.1056/NEJMoa1407222
9. Brentjens R J, Davila M L, Riviere I, Park J, Wang X, Cowell L G, et al. CD19-targeted T cells rapidly induce molecular remissions in adults with chemotherapy-refractory acute lymphoblastic leukemia. Sci Transl Med. 2013 Mar. 20; 5(177):177ra38. doi: 10.1126/scitranslmed.3005930.
10. Timmers M, Roex G, Wang Y, Campillo-Davo D, Van Tendeloo V F I, Chu Y, et al. Chimeric Antigen Receptor-Modified T Cell Therapy in Multiple Myeloma: Beyond B Cell Maturation Antigen. Front Immunol. 2019 Jul. 16; 10:1613. doi: 10.3389/fimmu.2019.01613.
11. Garfall A L, Fraietta J A, Maus M V. Immunotherapy with chimeric antigen receptors for multiple myeloma. Discovery medicine 2014; 17(91): 37-46.
12. Maus M V, June C H. CARTs on the road for myeloma. Clinical cancer research: an official journal of the American Association for Cancer Research 2014; 20(15): 3899-3901. doi: 10.1158/1078-0432.CCR-14-0721
13. Seckinger A, Delgado J A, Moser S, Moreno L, Neuber B, Grab A et al. Target Expression, Generation, Preclinical Activity, and Pharmacokinetics of the BCMA-T Cell Bispecific Antibody EM801 for Multiple Myeloma Treatment. Cancer cell 2017; 31(3): 396-410. doi: 10.1016/j.ccell.2017.02.002
14. Hipp S, Tai Y T, Blanset D, Deegen P, Wahl J, Thomas O et al. A novel BCMA/CD3 bispecific T-cell engager for the treatment of multiple myeloma induces selective lysis in vitro and in vivo. Leukemia 2017; 31(8): 1743-1751. doi: 10.1038/leu.2016.388
15. Ramadoss N S, Schulman A D, Choi S H, Rodgers D T, Kazane S A, Kim C H et al. An anti-B cell maturation antigen bispecific antibody for multiple myeloma. Journal of the American Chemical Society 2015; 137(16): 5288-5291. doi: 10.1021/jacs.5b01876
16. Ali S A, Shi V, Maric I, Wang M, Stroncek D F, Rose J J et al. T cells expressing an anti-B-cell maturation antigen chimeric antigen receptor cause remissions of multiple myeloma. Blood 2016; 128(13): 1688-1700. doi: 10.1182/blood-2016-04-711903
17. Brudno J N, Maric I, Hartman S D, Rose J J, Wang M, Lam N et al. T Cells Genetically Modified to Express an Anti-B-Cell Maturation Antigen Chimeric Antigen Receptor Cause Remissions of Poor-Prognosis Relapsed Multiple Myeloma. Journal of clinical oncology: official journal of the American Society of Clinical Oncology 2018; 36(22): 2267-2280. doi: 10.1200/JCO.2018.77.8084
18. Cohen A D, Garfall A L, Stadtmauer E A, Melenhorst J J, Lacey S F, Lancaster E, et al. B cell maturation antigen-specific CAR T cells are clinically active in multiple myeloma. J Clin Invest. 2019 Mar. 21; 129(6):2210-2221. doi: 10.1172/JCI126397.
19. Raje N, Berdeja J, Lin Y, Siegel D, Jagannath S, Madduri D, et al. Anti-BCMA CAR T-Cell Therapy bb2121 in Relapsed or Refractory Multiple Myeloma. N Engl J Med. 2019 May 2; 380(18):1726-1737. doi: 10.1056/NEJMoa1817226
20. Balmer J E, Blomhoff R. Gene expression regulation by retinoic acid. Journal of lipid research 2002; 43(11): 1773-1808.
21. Coltella N, Valsecchi R, Ponente M, Ponzoni M, Bernardi R. Synergistic Leukemia Eradication by Combined Treatment with Retinoic Acid and HIF Inhibition by EZN-2208 (PEG-SN38) in Preclinical Models of PML-RARalpha and PLZF-RARalpha-Driven Leukemia. Clinical cancer research: an official journal of the American Association for Cancer Research 2015; 21(16): 3685-3694. doi: 10.1158/1078-0432.CCR-14-3022
22. Chaudhari N, Talwar P, Lefebvre D'hellencourt C, Ravanan P. CDDO and ATRA Instigate Differentiation of IMR32 Human Neuroblastoma Cells. Frontiers in molecular neuroscience 2017; 10: 310. doi: 10.3389/fnmol.2017.00310
23. Martens J H, Brinkman A B, Simmer F, Francoijs K J, Nebbioso A, Ferrara F, et al. PML-RARalpha/RXR Alters the Epigenetic Landscape in Acute Promyelocytic Leukemia. Cancer Cell. 2010 Feb. 17; 17(2):173-85. doi: 10.1016/j.ccr.2009.12.042.
24. Mikesch J H, Gronemeyer H, So C W. Discovery of novel transcriptional and epigenetic targets in APL by global ChIP analyses: Emerging opportunity and challenge. Cancer Cell. 2010 Feb. 17; 17(2):112-4. doi: 10.1016/j.ccr.2010.01.012.
25. Nijhof I S, Casneuf T, van Velzen J, van Kessel B, Axel A E, Syed K et al. CD38 expression and complement inhibitors affect response and resistance to daratumumab therapy in myeloma. Blood 2016; 128(7): 959-970. doi: 10.1182/blood-2016-03-703439
26. Laurent S A, Hoffmann F S, Kuhn P H, Cheng Q, Chu Y, Schmidt-Supprian M et al. gamma-Secretase directly sheds the survival receptor BCMA from plasma cells. Nature communications 2015; 6: 7333. doi: 10.1038/ncomms8333
27. Sanchez E, Li M, Kitto A, Li J, Wang C S, Kirk D T et al. Serum B-cell maturation antigen is elevated in multiple myeloma and correlates with disease status and survival. British journal of haematology 2012; 158(6): 727-738. doi: 10.1111/j.1365-2141.2012.09241.x
28. Stone J D, Aggen D H, Schietinger A, Schreiber H, Kranz D M. A sensitivity scale for targeting T cells with chimeric antigen receptors (CARs) and bispecific T-cell Engagers (BiTEs). Oncoimmunology. 2012 Sep. 1; 1(6):863-873.
29. Nerreter T, Letschert S, Götz R, Doose S, Danhof S, Einsele H, et al. Super-resolution microscopy reveals ultra-low CD19 expression on myeloma cells that triggers elimination by CD19 CAR-T. Nat Commun. 2019 Jul. 17; 10(1):3137.
30. Walker A J, Majzner R G, Zhang L, Wanhainen K, Long A H, Nguyen S M, et al. Tumor Antigen and Receptor Densities Regulate Efficacy of a Chimeric Antigen Receptor Targeting Anaplastic Lymphoma Kinase. Mol Ther. 2017 Sep. 6; 25(9):2189-2201. doi: 10.1016/j.ymthe.2017.06.008.
31. Ramakrishna S, Highfill S L, Walsh Z, Nguyen S M, Lei H, Shern J F, et al. Modulation of Target Antigen Density Improves CAR T-cell Functionality and Persistence. Clin Cancer Res. 2019 Sep. 1; 25(17):5329-5341.
32. Fry T J, Shah N N, Orentas R J, Stetler-Stevenson M, Yuan C M, Ramakrishna S, et al. CD22-targeted CART cells induce remission in B-ALL that is naive or resistant to CD19-targeted CAR immunotherapy. Nat Med 2018; 24:20-8
33. Sotillo E, Barrett D M, Black K L, Bagashev A, Oldridge D, Wu G, et al. Convergence of acquired mutations and alternative splicing of CD19 enables resistance to CART-19 immunotherapy. Cancer Discov. 5, 1282-1295 (2015). doi: 10.1158/2159-8290
34. Grupp S A, Teachey D T, Porter D L, Grupp S A. Durable remissions in children with relapsed/refractory ALL Treated with T cells engineered with a CD19-targeted chimeric antigen receptor (CTL019). Blood 126, abstr. 681 (2015). doi: 10.1182/blood-2014-12-580068
35. Gardner R, Wu D, Cherian S, Fang M, Hanafi L A, Finney O, et al. Acquisition of a CD19-negative myeloid phenotype allows immune escape of MLL-rearranged B-ALL from CD19 CAR-T-cell therapy. Blood 127, 2406-2410 (2016). doi: 10.1182/blood-2015-08-665547
36. Salem D A, Maric I, Yuan C M, Liewehr D J, Venzon D J, Kochenderfer J et al. Quantification of B-cell maturation antigen, a target for novel chimeric antigen receptor T-cell therapy in Myeloma. Leukemia research 2018; 71: 106-111. doi: 10.1016/j.leukres.2018.07.015
37. Friedman K M, Garrett T E, Evans J W, Horton H M, Latimer H J, Seidel S L et al. Effective Targeting of Multiple B-Cell Maturation Antigen-Expressing Hematological Malignances by Anti-B-Cell Maturation Antigen Chimeric Antigen Receptor T Cells. Human gene therapy 2018; 29(5): 585-601. doi: 10.1089/hum.2018.001
38. Ruella M, Barrett D M, Kenderian S S, Shestova O, Hofmann T J, Perazzelli J et al. Dual CD19 and CD123 targeting prevents antigen-loss relapses after CD19-directed immunotherapies. The Journal of clinical investigation 2016; 126(10): 3814-3826. doi: 10.1172/JCI87366
39. Malavasi F. Editorial: CD38 and retinoids: a step toward a cure. Journal of leukocyte biology 2011; 90(2): 217-219. doi: 10.1189/jlb.0211069
40. Pont M J, Hill T, Cole G O, Abbott J J, Kelliher J, Salter A I, et al. γ-secretase inhibition increases efficacy of BCMA-specific chimeric antigen receptor T cells in multiple myeloma. Blood. 2019 Sep. 26. pii: blood.2019000050 doi: 10.1182/blood.2019000050.
41. Carpenter R O, Evbuomwan M O, Pittaluga S, Rose J J, Raffeld M, Yang S et al. B-cell maturation antigen is a promising target for adoptive T-cell therapy of multiple myeloma. Clinical cancer research: an official journal of the American Association for Cancer Research 2013; 19(8): 2048-2060. doi: 10.1158/1078-0432.CCR-12-2422
42. K Hofmann, et. al, Front. Immunol., 23 Apr. 2018, Targeting B Cells and Plasma Cells in Autoimmune Diseases, https://doi.org/10.3389/fimmu.2018.00835
43. A. Rubbert-Roth, et. al Efficacy and safety of various repeat treatment dosing regimens of rituximab in patients with active rheumatoid arthritis: results of a Phase III randomized study (MIRROR), Rheumatology, Volume 49, Issue 9, September 2010, Pages 1683-1693, https://doi.org/10.1093/rheumatology/keq116.
44. T. T. Smith, et. al, In situ programming of leukaemia-specific T cells using synthetic DNA nanocarriers, Nat Nanotechnol. 2017 August; 12(8): 813-820. Published online 2017 Apr. 17. doi: 10.1038/nnano.2017.57.
45. Agarwal S, et. al, Oncoimmunology. 2019 Oct. 10; 8(12):e1671761. In vivo generated human CAR T cells eradicate tumor cells. doi: 10.1080/2162402X.2019.1671761.
46. Liu E, et. al. Use of CAR-Transduced Natural Killer Cells in CD19-Positive Lymphoid Tumors. N Engl J Med. 2020 Feb. 6; 382(6):545-553. doi: 10.1056/NEJMoa1910607.

1-43. (canceled)

44. A method of treating a subject for cancer, comprising:
administering a retinoid and an immunotherapeutic anticancer agent capable of binding to BCMA to the patient, wherein the agent is a chimeric antigen receptor (CAR) capable or an antibody or fragment thereof.

45. The method of claim 44, wherein the subject has a hematological cancer.

46. The method of claim 45, wherein the subject has leukemia, lymphoma, or myeloma.

47. The method of claim 44, wherein the agent is a CAR.

48. The method of claim 44, wherein the agent is an antibody or antibody fragment.

49. The method of claim 48, wherein the antibody or antibody fragment is conjugated to a cytotoxic drug.

50. The method of claim 48, wherein the antibody or antibody fragment is bi-specific.

51. The method of claim 44, wherein the retinoid is a non-aromatic retinoid.

52. The method of claim 51, wherein the aromatic retinoid is an all-trans retinoic acid (ATRA), isotretionin (13-cis-retinoic acid), alitretinoin (9-cis-retinoic acid), retinal or retinol.

53. The method of claim 44, wherein the retinoid is an aromatic retinoid.

54. The method of claim 53, wherein the retinoid is selected from acitretin, etretinate and motretinid

55. The method of claim 44, wherein the retinoid is selected from adapalene, arotinoid, tazarotene, and bexarotene.

56. The method of claim 44, further comprising administering a gamma secretase inhibitor to the subject.

57. A method of treating a subject for an antibody-mediated autoimmune disease, comprising:
administering a retinoid and an immunotherapeutic anticancer agent capable of binding to BCMA to the patient, wherein the agent is a chimeric antigen receptor (CAR) capable or an antibody or fragment thereof.

58. The method of claim 57, wherein the antibody-mediated autoimmune disease is Graves' disease, myasthenia gravis, lupus erythematosus, rheumatoid arthritis, goodpasture syndrome, scleroderma, CREST syndrome, granulomatosis with polyangiitis, microscopic polyangiitis, pemphigus vulgaris, Sjögren's syndrome, diabetes mellitus type 1, primary biliary cholangitis, Hashimoto's thyreoiditis, neuromyelitis optica spectrum disorders, anti-NMDA receptor encephalitis, vasculitis or multiple sclerosis.

59. The method of claim 57, wherein the antibody or antibody fragment is conjugated to a cytotoxic drug and/or is bi-specific.

60. The method of claim 57, wherein the retinoid is a non-aromatic retinoid.

61. The method of claim 57, wherein the retinoid is an aromatic retinoid.

62. The method of claim 57, further comprising administering a gamma secretase inhibitor to the subject.

63. A kit comprising a retinoid and an immunotherapeutic anticancer agent capable of binding to BCMA to the patient.
